(19) United States
(12) Patent Application Publication
(10) Pub. No.: US 2022/0347232 A1
(43) Pub. Date: Nov. 3, 2022

(54) BACTERIAL COMPOSITIONS FOR THE TREATMENT OF DISEASE

(71) Applicants: Microbiotica Limited, Cambridge (GB); Genome Research Limited, Cambridge, Cambridgeshire (GB)

(72) Inventors: Trevor Lawley, Cambridge (GB); Anne Neville, Cambridge (GB); Samuel Forster, Cambridge (GB); Ian Holyer, Cambridge (GB); Dominika Klisko, Cambridge (GB); Seánin McCluskey, Cambridge (GB); Georgia Kapatai, Cambridge (GB); Nan Worrell, Cambridge (GB); Adam Wilkinson, Cambridge (GB)

(21) Appl. No.: 17/634,074

(22) PCT Filed: Aug. 14, 2020
(86) PCT No.: PCT/GB2020/051953

§ 371 Date: Feb. 9, 2022

(30) Foreign Application Priority Data
Aug. 15, 2019 (GB) 1911728.2

(51) Int. Cl. A61K 35/741; A61P 1/00; A61K 45/06; A61P 31/04

(52) U.S. Cl. CPC A61K 35/741; A61P 1/00; A61K 45/06; A61P 31/04

(57) ABSTRACT

The invention relates to bacterial compositions for the treatment or prevention thereof of a disease, in particular an infectious disease such as C. difficile infection.

### INTRODUCTION

Clostridium difficile, (more recently known as Clostridioides difficile, or C. difficile) is a Gram-positive rod-shaped, peritrichously flagellate, toxigenic, spore-forming bacterium that causes gastrointestinal disease with symptoms ranging from mild diarrhoea to severe, life-threatening colitis (1). Its spores are hardy, durable structures that enable effective transmission of this species across time and space, and between people. These spores are oxygen-resistant and are not killed by many healthcare disinfectants which means that rigorous cleaning is required for effective decontamination of the hospital and home environment (2).

As such, C. difficile is an eminent bacterial pathogen that has been described as an “immediate public health threat” that requires “ . . . urgent and aggressive action” by the US Centre for Disease Control in a recent report on the threat of antibiotic resistance in the United States (3). Hospitalised individuals, especially those that are immunocompromised or taking antibiotics, and the elderly are particularly susceptible to C. difficile disease, that may recur (1).

In the US, on the basis of surveillance at ten geographically distinct sites during 2011, the national burden of C. difficile disease was estimated at approximately half a million infections and was associated with ˜29,000 deaths that year alone (4). Another report from the US Centre for Disease Control and Prevention published in 2013, claimed that C. difficile disease causes 250,000 infections requiring hospitalisation and 14,000 deaths annually (3). In Europe, in 2016, more than 7000 C. difficile cases were reported across 20 different countries and, like in the USA, the majority of these infections were healthcare-associated (5). The cost of treating C. difficile disease is significant. The average recurrence rate of healthcare-associated C. difficile disease is ˜20% and the cost of care is typically driven up by the cost of treating recurrent disease (4). Interventions to prevent recurrent disease are therefore urgently sought to ease the economic burden of C. difficile infections (CDIs).

Traditionally, antibiotics and rehydration were the recommended first-line treatment for CDI, with vancomycin, metronidazole and fidaxomicin the antibiotics often prescribed to kill the pathogen (6). The emergence of antibiotic resistant C. difficile strains and the tendency of antibiotic treatments to underpin CDIs means that alternatives to antibiotics are urgently sought (3).

Faecal Microbiota Transplant (FMT), is a remarkably efficacious treatment for recurrent C. difficile disease and is strongly recommended on the basis of high-quality evidence from randomised controlled trials for the treatment of mild to severe recurrent CDIs (7). FMT is thought to work by restoring a diverse gut microbiome, and therefore colonisation resistance, to patients with a gut microbiome dysbiosis. As a treatment option, the main benefits of FMT include its tremendous and durable efficacy, an excellent short-term safety profile and high levels of patient satisfaction post-procedure (7). However, the precise mechanism(s) of action of FMT are not fully understood and very little long-term safety data is available.

Moreover, by its very nature, the composition of the raw material is not standardised and cannot be produced at scale. Furthermore, the stool is screened only for “known knowns” and any atypical or latent pathogens are not considered. Donor screening, stool processing (7) and delivery of FMT to patients also varies between clinics and has even been performed unsupervised, by lay-people in their own homes (9).

With the transition of FMT from an unorthodox procedure of last-resort to a reputable mainstream treatment option, inevitably, organisations and companies that seek to commercialise microbiome-based therapeutics have emerged. Stool banks now exist to supply FMT material to medical professionals and whole-stool based therapies for CDI and other indications, are under development as drugs (10). Indeed, even the therapeutic potential of loosely defined stool fractions, such as ethanol-resistant bacterial spores have been used successfully for the treatment of recurrent CDI (11). Although these stool-based therapies are produced by companies and organisations with a drug-development mindset, many of the drawbacks associated with FMT also apply to these products.

The logical progression from these undefined raw-stool based therapies are defined, rationally selected strains or consortia of purified and well characterised micro-organisms that can be used to successfully treat disease. These “Live Biotherapeutic Products” (LBPs) are biological products other than vaccines, that contain living organisms intended for the prevention, treatment or cure of diseases affecting humans (12). Although no LPBs have yet come to market, there are examples with proven efficacy for infectious disease indications including C. difficile, exist (13).

The advantages of the consortium approach apply mainly to aspects of product standardisation, safety and manufacturing. Unlike FMT, which is subject to compositional and quality variation; the strains that comprise a therapeutic consortium can be reliably reproduced to meet predefined specifications that can be analytically assessed. These therapeutic strains would have to be well-defined taxonomically, which means that it is realistic to expect that no ersatz or contaminating taxa would ever occur in the final product. Moreover, the chosen strains would also be well-characterised biologically, so that any potential for pathogenicity or genetic instability would be known. Understanding strain metabolism and behaviour enables the development of reproducible methods for large-scale production of the target strains, thereby facilitating manufacture of the LBP. Knowing the genotype and phenotype of the therapeutic strains underpins product safety and batch testing. It is also reasonable to assume that patients would be more receptive to LBPs delivered per os in tablet or capsule form because the mode of delivery would be less invasive and less disruptive than FMT, to their lifestyle.

Thus, there is a need to provide efficacious treatments for C. difficile infection and the present invention is aimed at addressing this need.

### SUMMARY OF THE INVENTION

The inventors have identified compositions comprising isolated bacteria which can be used to treat C. difficile infection. The invention therefore relates to therapeutic bacterial compositions each comprising a consortium of defined bacterial isolates which are useful in the treatment of disease, in particular in the treatment and prevention of C. difficile infection. The invention also relates to related methods of treatment and prevention of disease, in particular in the treatment and prevention of C. difficile infection.

In one aspect, the invention relates to a composition comprising two or more isolated bacteria selected from the following species: Bacteroides cellulosilyticus, Blautia sp., Coprococcus catus, Coprococcus comes, Dorea sp., Erysipelotrichaceae UCG-003 sp., Odoribacter splanchnicus, Parabacteroides distasonis, Ruminiclostridium 9 sp., Ruminococcus torques, Bacteroides fragilis, Blautia sp., Blautia sp., Lachnospiraceae FCS020 group sp., Lachnoclostridium sp., Bifidobacterium longum and Bacteroides sp. The composition may comprise or consist of 2, 3, 4, 5, 6, 7, 8, 9, 10, 11, 12, 13, 14, 15, 16 or 17 isolated bacteria selected from this list. In one embodiment, the two or more isolated bacteria comprise 16S rDNA sequences having at least 95%, for example 97%, 98%, 98.7% or 99% sequence identity with a nucleic acid sequence selected from SEQ ID NOs: 1 to 21. In one embodiment, the bacteria are lyophilized.

The invention also relates to a composition comprising two or more isolated bacteria wherein said bacteria are selected from a bacterium having a 16sDNA of SEQ ID No. 1, 2 or 3 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 4 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 5 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 6 or a 16S rDNA sequence having at least 90% e.g. at least 91%, 92%, 93%, 94%, 95%, 96%, 97%, 98%, 99%; e.g. 97% or 98.7% identity thereto; a bacterium. having a 16sDNA of SEQ ID No. 7 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 8 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 9 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of the following SEQ ID Nos. 10 or 11 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 12 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 13 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 14 or 15 or a 16S rDNA sequence having at least 90% e.g. at least 91%, 92%, 93%, 94%, 95%, 96%, 97%, 98%, 99%; e.g. 97% or 98.7% identity thereto, a bacterium having a 16sDNA of SEQ ID No. 16 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 17 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 18 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 19 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 20 or a 16S rDNA sequence having at least 90% e.g. at least 91%, 92%, 93%, 94%, 95%, 96%, 97%, 98%, 99%; e.g. 97% or 98.7% identity thereto and a bacterium having a 16sDNA of SEQ ID No. 21 or a 16S rDNA sequence having at least 90% e.g. at least 91%, 92%, 93%, 94%, 95%, 96%, 97%, 98%, 99%; e.g. 97% or 98.7% identity thereto.

The invention also provides a composition comprising one; e.g. two to 17 or more isolated strain deposited under an accession number provided herein.

The invention also relates to a method for treating or preventing a disease in a subject comprising administering a composition described herein.

In another aspect, the invention relates to a composition described herein for use in the treatment of disease.

According to the method and compositions, the disease to be treated a pathogenic infection, such as a C. difficile infection.

The invention also provides a kit comprising a bacterial composition described herein.

### DESCRIPTION OF FIGURES

FIG. 1. Survival graph. A composition (composition A) having 17 bacterial isolates was tested in an in vivo model as described in example 2. FMT therapy and a vehicle were also tested.

FIG. 2. Survival graph. A composition (composition B) having 15 bacterial isolates was tested in an in vivo model as described in example 2. Vehicle was also tested.

FIG. 3. Survival graph. A composition (composition C) having 10 bacterial isolates was tested in an in vivo model as described in example 2. Vehicle was also tested.

FIG. 4. 4a) Average weight loss two days post C. difficile challenge, as a percentage of the average weight of the mice immediately prior to C. difficile challenge, of mice treated with a 17 strain consortium of bacterial strains (composition A) compared to mice treated with vehicle (placebo). 4b) Average weight loss two days post C. difficile challenge, as a percentage of the average weight of the mice immediately prior to C. difficile challenge, of mice treated with a consortium of 10 bacterial strains (composition C) compared to mice treated with vehicle (placebo). 4c) Average weight loss two days post C. difficile challenge, as a percentage of the average weight of the mice immediately prior to C. difficile challenge, of mice treated with a consortium of 4 bacterial strains (composition D) compared to mice treated with vehicle (placebo).

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of microbiology, cell and tissue culture, pathology, molecular biology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

The nomenclatures used in connection with, and the laboratory procedures and techniques of analytical chemistry, microbiology, bioinformatics and medicinal and pharmaceutical chemistry described herein are those well-known and commonly used in the art.

The invention relates to therapeutic bacterial compositions each comprising a consortium of defined bacterial isolates. The compositions are useful in the treatment of disease. Thus, the compositions are not faecal microbiota transplants (FMT) and do not contain faecal material, but contain defined mixtures of bacterial isolates free of faecal material. FMTs usually consist of a stool sample from a healthy human donor which is administered directly to the recipient, e.g. in the form of an enema, without bacteria present in the stool sample being isolated prior to the administration of the FMT to the recipient. An advantage of the present composition is therefore that it comprises no undefined components, which are present in FMTs, thereby allowing the therapeutic composition to be standardised and increasing safety of the composition.

The term “isolated” refers to bacteria that are isolated from its natural environment. The isolated bacteria, e.g. isolated bacterial strains, are substantially free of other cellular material, chemicals and/or faecal material.

Thus, as used herein, the term “isolated” bacteria refers to bacteria that have been separated from one or more undesired component, such as another bacterium or bacterial strain, one or more component of a growth medium, and/or one or more component of a sample, such as a faecal sample. In some embodiments, the bacteria are substantially isolated from a source such that other components of the source are not detected. As used herein, the term “species” refers to a taxonomic entity as conventionally defined by genomic sequence and phenotypic characteristics. A “strain” is a particular instance of a species that has been isolated and purified according to conventional microbiological techniques.

In one embodiment, the bacteria of the composition are inactive prior to administration. For example, the bacteria are lyophilised. In one embodiment, the composition includes vegetative bacterial cells and does not include bacterial spores. In one embodiment, the composition includes vegetative bacterial cells and/or bacterial spores. In one embodiment, the composition includes vegetative bacterial cells and does not include bacterial spores or is substantially devoid of spores. In one embodiment, the composition includes fewer than about 0.5%, 1%, 2%, 3%, 4% or 5% spores. In one embodiment, the composition includes vegetative bacterial cells and/or bacterial spores.

The composition is preferably a live bacterial therapeutic, bacteriotherapy or a live biotherapeutic product. As described herein, a live bacterial product (also referred to as a bacterial composition, live bacterial consortium, or bacterial consortium) comprises one or more bacterial strains from one or more bacterial species as described herein. The live bacterial product provides a Live Bacterial Therapy (LBT). The term Live Bacterial Therapy or LBT is interchangeably used with bacteriotherapy herein and defines a therapy using live bacteria to restore health or cure disease.

In one embodiment, the composition may be as described above, but does not comprise bacteria of any other species, i.e. species not listed in Table 1a, or the composition comprises only de minimis or biologically irrelevant amounts of bacteria from another species. By biologically irrelevant is meant bacteria that do not have an effect on the treatment of C. difficile infection.

In one embodiment, the isolated bacteria, e.g. isolated bacterial strains, may be viable bacteria that are capable of colonising the gastrointestinal gut of a subject when administered to said subject.

In a first aspect, the invention relates to a composition comprising two or more isolated bacteria wherein said bacteria are selected from a bacterium having a 16sDNA of SEQ ID No. 1, 2 or 3 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 4 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 5 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 6 or a 16S rDNA sequence having at least 90% e.g. at least 91%, 92%, 93%, 94%, 95%, 96%, 97%, 98%, 99%; e.g. 97% or 98.7% identity thereto; a bacterium. having a 16sDNA of SEQ ID No. 7 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 8 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 9 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of the following SEQ ID Nos. 10 or 11 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 12 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 13 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 14 or 15 or a 16S rDNA sequence having at least 90% e.g. at least 91%, 92%, 93%, 94%, 95%, 96%, 97%, 98%, 99%; e.g. 97% or 98.7% identity thereto, a bacterium having a 16sDNA of SEQ ID No. 16 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 17 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 18 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 19 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 20 or a 16S rDNA sequence having at least 90% e.g. at least 91%, 92%, 93%, 94%, 95%, 96%, 97%, 98%, 99%; e.g. 97% or 98.7% identity thereto and a bacterium having a 16sDNA of SEQ ID No. 21 or a 16S rDNA sequence having at least 90% e.g. at least 91%, 92%, 93%, 94%, 95%, 96%, 97%, 98%, 99%; e.g. 97% or 98.7% identity thereto.

Table 1a below lists 17 different bacterial species. The name of the closest bacterial species identified is provided and possible alternative name and/or closely related species based on closely related species from public databases are also given. Isolated bacteria present in the compositions of the invention are selected from the 17 species below. The taxonomic name is provided as well as exemplary 16S rDNA sequence for each species. It should be appreciated that SEQ ID NOs: 1-21 may include full length or partial 16S rDNA sequences. Also, as explained further below, the bacteria may have a 16S rDNA sequence with certain sequence identity to the SEQ ID listed below. As can be seen below, for the species listed in rows 1, 8 and 11, different exemplary sequences are provided.

TABLE 1a

| | | | Possible alterna- |
|---|---|---|---|
| | | | tive name and/or |
| | | | closely related |
| | | 16S rDNA | species based on |
| | | sequence - | closely related |
| Species | Taxonomy- closest | sequence | species from pub- |
| Number | bacterial species | identifier | lic databases |
| 1 | Bacteroides | SEQ ID NO. 1 | n/a |
| | cellulosilyticus | SEQ ID NO. 2 | |
| | | SEQ ID NO. 3 | |
| 2 | Blautia sp. | SEQ ID NO. 4 | n/a |
| 3 | Coprococcus catus | SEQ ID NO. 5 | n/a |
| 4 | Coprococcus comes | SEQ ID NO. 6 | n/a |
| 5 | Dorea sp. | SEQ ID NO. 7 | Dorea |
| | | | formicigenerans |
| 6 | Erysipelotrichaceae | SEQ ID NO. 8 | n/a |
| | UCG-003 sp. | | |
| 7 | Odoribacter | SEQ ID NO. 9 | n/a |
| | splanchnicus | | |
| 8 | Parabacteroides | SEQ ID NO. 10 | n/a |
| | distasonis | SEQ ID NO. 11 | |
| 9 | Ruminiclostridium | SEQ ID NO. 12 | Anaerostipes hadrus |
| | 9 sp. | | |
| 10 | Ruminococcus torques | SEQ ID NO. 13 | Lachnospiraciae |
| | | | bacterium |
| 11 | Bacteroides fragilis | SEQ ID NO. 14 | n/a |
| | | SEQ ID NO. 15 | |
| 12 | Blautia sp. | SEQ ID NO. 16 | n/a |
| 13 | Blautia sp. | SEQ ID NO. 17 | n/a |
| 14 | Lachnospiraceae | SEQ ID NO. 18 | n/a |
| | FCS020 group sp. | | |
| 15 | Lachnoclostridium sp. | SEQ ID NO. 19 | Clostridium |
| | | | citroniae |
| 16 | Bifidobacterium | SEQ ID NO. 20 | n/a |
| | longum | | |
| 17 | Bacteroides sp. | SEQ ID NO. 21 | Bacteroides |
| | | | xylanisolvens |

Table 1b lists exemplary strains of the bacteria in table 1a. Compositions comprising such strains are within the scope of the invention. The bacteria were deposited under the Budapest Treaty on the International Recognition of the Deposit of Microorganisms for the Purposes of Patent Procedures at the Leibniz-Institut DSMZ—Deutsche Sammlung von Mikroorganismen and Zellkulturen GmbH (DSMZ), Inhoffenstr. 7B, 38124 Braunschweig by Microbiotica Limited. The accession number, deposit date and Depositor's reference number shown below.

| Depositor's | DSMZ accession | | | |
|---|---|---|---|---|
| reference | number for | Date of Deposition | Taxonomy- closest | 16S rDNA sequence - |
| number | bacterial Strain | at DSMZ | bacterial species | sequence identifier |
| MCC701 | DSM 33265 | 13^{th} Sep. 2019 | Bacteroides cellulosilyticus | SEQ ID NO. 1 |
| MCC136 | DSM 33263 | 13^{th} Sep. 2019 | Bacteroides cellulosilyticus | SEQ ID NO. 2 |
| MCC2 | DSM 33261 | 13^{th} Sep. 2019 | Bacteroides cellulosilyticus | SEQ ID NO. 3 |
| MCC702 | DSM 33266 | 13^{th} Sep. 2019 | Blautia sp. | SEQ ID NO. 4 |
| MCC703 | DSM 33277 | 13^{th} Sep. 2019 | Coprococcus catus | SEQ ID NO. 5 |
| MCC704 | DSM 33278 | 13^{th} Sep. 2019 | Coprococcus comes | SEQ ID NO. 6 |
| MCC705 | DSM 33267 | 13^{th} Sep. 2019 | Dorea sp. | SEQ ID NO. 7 |
| MCC706 | DSM 33268 | 13^{th} Sep. 2019 | Erysipelotrichaceae UCG-003 sp. | SEQ ID NO. 8 |
| MCC707 | DSM 33269 | 13^{th} Sep. 2019 | Odoribacter splanchnicus | SEQ ID NO. 9 |
| MCC708 | DSM 33270 | 13^{th} Sep. 2019 | Parabacteroides distasonis | SEQ ID NO. 10 |
| MCC173 | DSM 33264 | 13^{th} Sep. 2019 | Parabacteroides distasonis | SEQ ID NO. 11 |
| MCC709 | DSM 33271 | 13^{th} Sep. 2019 | Ruminiclostridium 9 sp. | SEQ ID NO. 12 |
| MCC710 | DSM 33279 | 13^{th} Sep. 2019 | Ruminococcus torques | SEQ ID NO. 13 |
| MCC711 | DSM 33272 | 13^{th} Sep. 2019 | Bacteroides fragilis | SEQ ID NO. 14 |
| MCC93 | DSM 33262 | 13^{th} Sep. 2019 | Bacteroides fragilis | SEQ ID NO. 15 |
| MCC712 | DSM 33282 | 13^{th} Sep. 2019 | Blautia sp. | SEQ ID NO. 16 |
| MCC713 | DSM 33283 | 13^{th} Sep. 2019 | Blautia sp. | SEQ ID NO. 17 |
| MCC714 | DSM 33280 | 13^{th} Sep. 2019 | Lachnospiraceae FCS020 group sp. | SEQ ID NO. 18 |
| MCC715 | DSM 33281 | 13^{th} Sep. 2019 | Lachnoclostridium sp. | SEQ ID NO. 19 |
| MCC716 | DSM 33273 | 13^{th} Sep. 2019 | Bifidobacterium longum | SEQ ID NO. 20 |
| MCC717 | DSM 33274 | 13^{th} Sep. 2019 | Bacteroides sp. | SEQ ID NO. 21 |

In one embodiment, the composition comprises or consists of 17 isolated bacteria, e.g. bacteria from each of the different 17 species listed in table 1a, for example with reference to the sequences as shown in the table or a sequence with identity thereto as explained below. In one embodiment, the bacteria are defined by reference to the sequence shown in table 1a. For the species listed in rows 1, 8 and 11, different exemplary sequences are provided. One or more of these may be included in the composition. In one embodiment, the bacteria are selected from the strains in table 1b.

As will be apparent to a skilled person, different bacteria selected from those listed in table 1 can be combined in a single composition. For example, the composition comprises or consists of at least 2, e.g. up to 3, up to 4, up to 5, up to 6, up to 7, up to 8, up to 9, up to 10, up to 11, up to 12, up to 13, up to 14, up to 15, up to 16 or up to 17 isolated bacteria selected from those shown in table 1a or the strains in 1b, for example with reference to the sequences as shown in the table.

In one embodiment, the composition comprises or consists of 2, 3, 4, 5, 6, 7, 8, 9, 10, 11, 12, 13, 14, 15, 16 or 17 isolated bacteria selected from those listed in table 1a or the strains in 1b, for example with reference to the sequences as shown in the table.

In one embodiment, the composition comprises or consists of 4, 5, 7, 10 or 15 isolated bacteria selected from those shown in table 1a or the strains in 1b, for example with reference to the sequences as shown in the table.

In one embodiment, the composition comprises or consists of at least 2, at least 3, at least 4, at least 5, at least 6, at least 7, at least 8, at least 9, at least 10, at least 11, at least 12, at least 13, at least 14, at least 15, at least 16 or at least 17 isolated bacteria selected from those listed in table 1a or the strains in 1b, for example with reference to the sequences as shown in the table.

In one embodiment, the composition comprises no more than 4, 7, 10 or 15 isolated bacteria selected from those shown in table 1a or the strains in 1b, for example with reference to the sequences as shown in the table.

In one embodiment, the composition comprises or consists of 2 to 4, 2 to 5, 2 to 6, 2 to 7, 2 to 8, 2 to 9, 2 to 10, 2 to 11, 2 to 12, 2 to 13, 2 to 14, 2 to 15, 2 to 16 or 2 to 17 isolated bacteria selected from those shown in table 1, for example with reference to the sequences as shown in table 1a or the strains in 1b.

In one embodiment, the composition comprises an isolated bacterial mixture consisting of 2 to 17 isolated bacteria (e.g. 3, 4, 5, 6, 7, 8, 9, 10, 11, 12, 13, 14, 15, 16, 17 isolated bacteria) having at least 97% sequence identity to 16s DNA sequences selected from SEQ ID Nos 1 to 21. Exemplary compositions are set out herein, but all combinations are within the scope of the invention.

In one embodiment, the composition comprises or consists of isolated bacteria selected from the following species: Bacteroides cellulosilyticus (e.g. SEQ ID Nos 1, 2 or 3), Parabacteroides distasonis (e.g. SEQ ID Nos 10 or 11), Bacteroides fragilis (e.g. SEQ ID Nos 1, 2 or 3) and Bacteroides sp. (e.g. SEQ ID Nos 14 or 15).

In one embodiment, the composition comprises or consists of isolated bacteria as shown for composition A, B, C or D (see examples).

A skilled person would appreciate that the bacterial species selected from Table 1a or the strains in 1b and for use in the composition and methods of the invention can have the sequence shown in Table 1a or the strains in 1b or a sequence that has certain percentage identity thereto and retains biological activity; i.e. efficacy against C. difficile infection when used in the compositions described herein.

Methods of determining sequence identity are known in the art. It is known that clades, operational taxonomic units (OTUs), species, and strains are, in some embodiments, identified by their 16S rDNA sequence. The relatedness can be determined by the percent identity and this can be determined using methods known in the art.

Bacterial species and strains used in a composition as described herein are identified based on the 16S nucleic acid sequence (full length or part thereof, such as V regions). The 16S ribosomal RNA gene codes for the RNA component of the 30S subunit of the bacterial ribosome. It is widely present in all bacterial species. Different bacterial species have one to multiple copies of the 16S rRNA gene. 16S rRNA gene sequencing is by far one of the most common methods targeting housekeeping genes to study bacterial phylogeny and genus/species classification. Thus, bacteria can be taxonomically classified based on the sequence of the gene encoding the 16S nucleic acid sequence, e.g. ribosomal RNA (rRNA) in the bacterium. This gene sequence is also referred to as the ribosomal DNA sequence (rDNA). The bacterial 16S rDNA is approximately 1500 nucleotides in length. The V1-V9 regions of the 16S refer to the first nine hypervariable regions of the 16S rRNA gene that are often used for genetic typing of bacterial samples. In some embodiments, at least one of V1 to V9 is used to characterise the bacterial isolate.

As used herein, the term “homology” or “identity” generally refers to the percentage of nucleic acid residues in a sequence that are identical with the residues of the reference sequence with which it is compared, after aligning the sequences and in some embodiments after introducing gaps, if necessary, to achieve the maximum percentage homology, and not considering any conservative substitutions as part of the sequence identity. Thus, the percentage homology between two nucleic acid sequences is equivalent to the percentage identity between the two sequences. Methods and computer programs for the alignment are well known. The percentage identity between two sequences can be determined using well known mathematical algorithms. References to a percentage sequence identity between two nucleotide sequences means that, when aligned, that percentage of nucleotides are the same in comparing the two sequences. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence as provided herein.

In one embodiment, the degree of sequence identity between a query sequence and a reference sequence can be determined with the aid of a commercially available sequence comparison program. This typically involves aligning the two sequences using the default scoring matrix and default gap penalty, identifying the number of exact matches, and dividing the number of exact matches with the length of the reference sequence. Suitable computer programs useful for determining identity include, for example, BLAST (blast.ncbi.nlm.nih.gov). One alignment is determined by the Smith-Waterman homology search algorithm or the Needleman-Wunsch algorithm. In yet a further embodiment, the global alignment program is selected from the group consisting of EMBOSS Needle and EMBOSS stretcher and the sequence identity is calculated by identifying the number of exact matches identified by the program divided by the “alignment length,” where the alignment length is the length of the entire alignment including gaps and overhanging parts of the sequences.

The full length or partial 16S rDNA of the bacterial species listed in table 1 and which are used in the compositions and methods of the invention thus has at least 90% e.g. at least 91%, 92%, 93%, 94%, 95%, 96%, 97%, 98%, 99% or 100%, sequence identity to the corresponding 16S rDNA as listed in table 1 (i.e. SEQ IDs 1 to 21). In one embodiment, said sequence identity is at least 95%. In one embodiment, said sequence identity is at least 97%. In one embodiment, said sequence identity is at least 98.7%.

In one aspect, the composition therefore comprises two or more bacteria comprising a 16S rDNA sequence selected from SEQ ID. NO. 1 to 21 or comprising a 16S rDNA sequence having at least 90% e.g. at least 91%, 92%, 93%, 94%, 95%, 96%, 97%, 98%, 99%; e.g. 97% or 98.7% identity to a nucleic acid sequence selected from SEQ ID NOs. 1 to 21. In one embodiment, a bacterium present in the composition belongs to the same species as a bacterium disclosed herein, has at least 90% e.g. at least 91%, 92%, 93%, 94%, 95%, 96%, 97%, 98%, 99%; e.g. 97% or 98.7% identity to a nucleic acid sequence selected from SEQ ID NOs. 1 to 21 and retains activity against C. difficile infection. Activity against C. difficile infection can be assessed using an vitro, ex vivo or in vivo assessment, for example using a murine model as described in the examples.

In one embodiment, the composition comprises or consists of bacteria from 17 different species having a 16sDNA of a SEQ selected from ID Nos. 1 to 21 or a 16S rDNA sequence having at least 90% e.g. at least 91%, 92%, 93%, 94%, 95%, 96%, 97%, 98%, 99%; e.g. 97% or 98.7% identity thereto. As explained above, for some species, more than one sequence is provided. In one embodiment, SEQ ID NO. 1 or a 16S rDNA sequence having at least 90% e.g. at least 91%, 92%, 93%, 94%, 95%, 96%, 97%, 98%, 99%; e.g. 97% or 98.7% identity thereto is selected. In one embodiment, SEQ ID NO. 10 or a 16S rDNA sequence having at least 90% e.g. at least 91%, 92%, 93%, 94%, 95%, 96%, 97%, 98%, 99%; e.g. 97% or 98.7% identity thereto is selected. In one embodiment, SEQ ID NO. 14 or a 16S rDNA sequence having at least 90% e.g. at least 91%, 92%, 93%, 94%, 95%, 96%, 97%, 98%, 99%; e.g. 97% or 98.7% identity thereto is selected. Thus, the composition comprises or consists of bacteria selected from SEQ ID NO. 1, 4, 5, 6, 7, 8, 9, 10, 12, 13, 14, 16, 17, 18, 19, 20, 21 or a sequence having at least 90% e.g. at least 91%, 92%, 93%, 94%, 95%, 96%, 97%, 98%, 99%; e.g. 97% or 98.7% identity thereto.

In one embodiment, the composition comprises or consists of the following 15 bacteria, e.g. from 15 different species, having a 16sDNA of the following SEQ ID Nos.:

SEQ ID No. 1 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 6 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 7 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 8 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 9 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 10 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 12 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 13 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 14 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 16 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 17 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 18 or a 16S rDNA sequence having at least 90% e.g. at least 91%, 92%, 93%, 94%, 95%, 96%, 97%, 98%, 99%; e.g. 97% or 98.7% identity thereto and

SEQ ID No. 19 or a 16S rDNA sequence having at least 90% e.g. at least 91%, 92%, 93%, 94%, 95%, 96%, 97%, 98%, 99%; e.g. 97% or 98.7% identity thereto.

In one embodiment, SEQ ID NO. 1 may be replaced with SEQ ID No. 2 or 3 or a 16S rDNA sequence having at least 90% e.g. at least 91%, 92%, 93%, 94%, 95%, 96%, 97%, 98%, 99%; e.g. 97% or 98.7% identity thereto; SEQ ID NO. 10 may be replaced with SEQ ID No. 11 or a 16S rDNA sequence having at least 90% e.g. at least 91%, 92%, 93%, 94%, 95%, 96%, 97%, 98%, 99%; e.g. 97% or 98.7% identity thereto and SEQ ID NO. 14 may be replaced with SEQ ID No. 15 or a 16S rDNA sequence having at least 90% e.g. at least 91%, 92%, 93%, 94%, 95%, 96%, 97%, 98%, 99%; e.g. 97% or 98.7% identity thereto.

In another embodiment, the composition comprises or consists of the following 10 bacteria having a 16sDNA of the following SEQ ID Nos.:

SEQ ID No. 1 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 6 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 7 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 8 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 9 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 10 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 12 or a 16S rDNA sequence having at least 90% e.g. at least 91%, 92%, 93%, 94%, 95%, 96%, 97%, 98%, 99%; e.g. 97% or 98.7% identity thereto and

SEQ ID No. 13 or a 16S rDNA sequence having at least 90% e.g. at least 91%, 92%, 93%, 94%, 95%, 96%, 97%, 98%, 99%; e.g. 97% or 98.7% identity thereto.

In one embodiment, SEQ ID NO. 1 may be replaced with SEQ ID No. 2 or 3 or a 16S rDNA sequence having at least 90% e.g. at least 91%, 92%, 93%, 94%, 95%, 96%, 97%, 98%, 99%; e.g. 97% or 98.7% identity thereto; SEQ ID NO. 10 may be replaced with SEQ ID No. 11 or a 16S rDNA sequence having at least 90% e.g. at least 91%, 92%, 93%, 94%, 95%, 96%, 97%, 98%, 99%; e.g. 97% or 98.7% identity thereto.

In another embodiment, the composition comprises or consists of the following 7 bacteria having a 16sDNA of SEQ ID Nos.:

SEQ ID No. 1 or a 16S rDNA sequence having at least 90% e.g. at least 91%, 92%, 93%, 94%, 95%, 96%, 97%, 98%, 99%; e.g. 97% or 98.7% identity thereto

SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 6 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 7 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 8 or a 16S rDNA sequence having at least 90% e.g. at least 91%, 92%, 93%, 94%, 95%, 96%, 97%, 98%, 99%; e.g. 97% or 98.7% identity thereto and

SEQ ID No. 9 or a 16S rDNA sequence having at least 90% e.g. at least 91%, 92%, 93%, 94%, 95%, 96%, 97%, 98%, 99%; e.g. 97% or 98.7% identity thereto.

In one embodiment, SEQ ID NO. 1 may be replaced with SEQ ID No. 2 or 3 or a 16S rDNA sequence having at least 90% e.g. at least 91%, 92%, 93%, 94%, 95%, 96%, 97%, 98%, 99%; e.g. 97% or 98.7% identity thereto.

In another embodiment, the composition comprises or consists of the following 5 bacteria having a 16sDNA of SEQ ID Nos.:

SEQ ID No. 1 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 6 or a 16S rDNA sequence having at least 90% e.g. at least 91%, 92%, 93%, 94%, 95%, 96%, 97%, 98%, 99%; e.g. 97% or 98.7% identity thereto and

SEQ ID No. 7 or a 16S rDNA sequence having at least 90% e.g. at least 91%, 92%, 93%, 94%, 95%, 96%, 97%, 98%, 99%; e.g. 97% or 98.7% identity thereto.

In one embodiment, SEQ ID NO. 1 may be replaced with SEQ ID No. 2 or 3 or a 16S rDNA sequence having at least 90% e.g. at least 91%, 92%, 93%, 94%, 95%, 96%, 97%, 98%, 99%; e.g. 97% or 98.7% identity thereto.

In another embodiment, the composition comprises or consists of the following 4 bacteria having a 16sDNA of SEQ ID Nos.:

SEQ ID No. 3 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 11 or a 16S rDNA sequence having at least 90% e.g. at least 91%, 92%, 93%, 94%, 95%, 96%, 97%, 98%, 99%; e.g. 97% or 98.7% identity thereto,

SEQ ID No. 15 or a 16S rDNA sequence having at least 90% e.g. at least 91%, 92%, 93%, 94%, 95%, 96%, 97%, 98%, 99%; e.g. 97% or 98.7% identity thereto and

SEQ ID No. 21 or a 16S rDNA sequence having at least 90% e.g. at least 91%, 92%, 93%, 94%, 95%, 96%, 97%, 98%, 99%; e.g. 97% or 98.7% identity thereto.

In one embodiment, SEQ ID NO. 3 may be replaced with SEQ ID No. 1 or 2 or a 16S rDNA sequence having at least 90% e.g. at least 91%, 92%, 93%, 94%, 95%, 96%, 97%, 98%, 99%; e.g. 97% or 98.7% identity thereto; SEQ ID NO. 11 may be replaced with SEQ ID No. 10 or a 16S rDNA sequence having at least 90% e.g. at least 91%, 92%, 93%, 94%, 95%, 96%, 97%, 98%, 99%; e.g. 97% or 98.7% identity thereto and SEQ ID NO. 15 may be replaced with SEQ ID No. 14 or a 16S rDNA sequence having at least 90% e.g. at least 91%, 92%, 93%, 94%, 95%, 96%, 97%, 98%, 99%; e.g. 97% or 98.7% identity thereto.

In one embodiment, the composition is selected from composition B, C or D as shown in tables 2, 3 or 4, e.g. bacteria having a species or sequence as shown for these compositions. In one embodiment, the composition is selected from composition B, C or D as shown in tables 2, 3 or 4 wherein the bacteria of the composition have a 16sDNA of SEQ ID Nos as provided in the respective table or a or a 16S rDNA sequence having at least 90% e.g. at least 91%, 92%, 93%, 94%, 95%, 96%, 97%, 98%, 99%; e.g. 97% or 98.7% identity thereto.

In one example, species used in the composition are identified based on their 16S rDNA sequence (e.g., full-length sequence, or partial sequence). In some cases, strains of bacterial species useful in an invention, e.g., strains of the species disclosed herein, can be obtained from a public biological resource center such as the ATCC (atcc.org), the DSMZ (dsmz.de), or the Riken BioResource Center (en.brc.riken.jp). 16s rDNA sequences useful for identifying species or other aspects of the invention can be obtained from public databases, e.g., the Human Microbiome Project (HMP) web site or GenBank.

A skilled person would appreciate that the compositions described herein can include any strain. A skilled person would appreciate that the compositions may include one or more than one strain of a particular bacterial species as listed in table. For example, the bacterial strains having 16S rDNA sequences with nucleic acid sequences SEQ ID NOs. 1, 2 or 3 all have Bacteroides cellulosilyticus as the related taxonomic species designation. The bacterial strains having 16S rDNA sequences with nucleic acid sequences SEQ ID NOs. 10 or 11 all have Parabacteroides distasonis as the related taxonomic species designation. The bacterial strains having 16S rDNA sequences with nucleic acid sequences SEQ ID NOs. 14 or 15 all have Bacteroides fragilis as the related taxonomic species designation. In one embodiment, the strain is selected from one of the strains in Table 1b.

In one embodiment, the compositions comprise the bacterial species listed herein and do not include further bacterial species. However, the composition may further comprise pharmaceutical excipients. In one embodiment, the bacterial composition comprises or consists of the bacteria as listed in the examples. In one embodiment, the bacterial composition comprises or consists of the bacterial strains as listed in the examples.

In one embodiment, the bacteria of the composition are capable of colonising the gastrointestinal tract of a subject. In one embodiment, the bacteria of the composition are capable of sustained engraftment in the gastrointestinal tract of a subject.

In one embodiment, the composition also has one or more of the following characteristics:
The composition is effective in treating and/or preventing C. difficile infection in a subject or animal model;
The bacterial isolates of the composition displace C. difficile thereby treating infection in a subject;
The bacterial isolates reduce C. difficile colonisation of the gastrointestinal tract of a subject thereby treating infection;
The bacterial isolates of the composition colonise the gastrointestinal tract of the subject faster than C. difficile and therefore prevent the pathogen from colonising or recolonising the gastrointestinal tract;
The bacterial isolates of the composition confer colonisation resistance towards C. difficile thereby preventing recurrence of the disease in a subject;
The bacterial isolates of the composition neutralise the C. difficile toxin thereby preventing disease manifestation in a subject;
The bacterial isolates of the composition reduce the immunostimulatory effects of C. difficile thereby preventing disease manifestation in a subject;
The bacterial isolates of the composition reduce the rate of sporulation by C. difficile and/or spore shedding thereby limiting onward transmission and infection of a subject;
The bacterial isolates of the composition prevent C. difficile spore germination thereby preventing infection of a subjects;
The bacterial isolates of the composition reduce C. difficile growth and/or C. difficile survival in in vitro and in vivo models;
The bacterial isolates of the composition increase survival rates of mice challenged with C. difficile (as shown in the examples) and/or
The bacterial isolates of the composition provide increased protection from C. difficile infection-related weight loss mice challenged with C. difficile (as shown in the examples).

The characteristics listed above may be assessed using methods known to the skilled person. Exemplary assays are shown in the examples. In one embodiment, the composition is effective in treating and/or preventing C. difficile infection, reducing C. difficile growth and/or survival and the assay is an in vivo in a survival model of C. difficile infection or a weight loss a survival model of C. difficile infection. The survival model may be described as in Warn et al (14).

Thus, C. difficile infection can be assessed in suitable animal model, e.g. mice models as shown in the examples, and evaluated by studying survival and/or weight loss.

The bacterial isolates used in the composition are generally isolated from one or more healthy subject.

In some embodiments, one or more of the bacterial strains are human-derived bacteria, meaning the one or more bacterial strains were obtained from or identified from a human or a sample therefrom (e.g., a human donor). In some embodiments of the compositions provided herein, all of the bacterial strains are human-derived bacteria. In some embodiments of the compositions provided herein, the bacterial strains are derived from more than one human donor.

The bacterial strains used in the live bacterial products provided herein generally are isolated from the microbiome of healthy individuals, but in some cases may not be from healthy individuals. In some embodiments, the live bacterial products include strains originating from a single individual. In some embodiments, the live bacterial products include strains originating from multiple individuals. In some embodiments, the bacterial strains are obtained from multiple individuals, isolated and grown up individually. The bacterial compositions that are grown up individually may subsequently be combined to provide the compositions of the disclosure. It should be appreciated that the origin of the bacterial strains of the live bacterial products provided herein is not limited to the human microbiome from a healthy individual.

The bacterial isolates can be tested as described in WO2013/171515. In one embodiment, bacterial strains are cultured and grown individually and then combined in the composition.

A bacterial isolate used in the composition is preferably a non-pathogenic strain. In other words, the bacterium preferably does not cause a disease in a healthy human individual when administered to said individual.

In one embodiment, each bacterium present in the composition is susceptible to treatment with one or more antibiotics. In other words, the bacterium is not resistant to treatment with at least one antibiotic. This allows antibiotic treatment of an individual in the event that one or more of the bacteria included in a therapeutic composition administered to the individual cause disease in the individual, contrary to expectations. Thus, in one embodiment, the bacterium is susceptible to treatment with one or more antibiotics selected from the group consisting of: a beta-lactam, fusidic acid, elfamycin, aminoglycoside, fosfomycin, tunicamycin metronidazole and/or vancomycin. In vitro and in silico methods for screening bacteria for antibiotic resistance are known in the art.

In one embodiment, the isolated bacterium included in the compositions may not comprise one or more genes encoding one or more virulence factors and/or preferably does not produce one or more virulence factors. Virulence factors in this context are properties which enhance the potential of a bacterium to cause disease in an individual. Virulence factors include the production of bacterial toxins, such as endotoxins and exotoxins by a bacterium, as well as the production of hydrolytic enzymes that may contribute to the pathogenicity of the bacterium. Methods for screening bacteria for genes encoding virulence factors are known in the art.

The various aspects of the invention, that is the compositions an, methods, kits and uses, also encompass one or more strain deposited under the Budapest Treaty on the International Recognition of the Deposit of Microorganisms for the Purposes of Patent Procedures at the DSMZ. Specifically, in one aspect, the therapeutic compositions of the present invention comprise at least one isolated bacterial strain, wherein the bacterium is a bacterium as deposited under the Budapest Treaty on the International Recognition of the Deposit of Microorganisms for the Purposes of Patent Procedures at the Leibniz-Institut DSMZ—Deutsche Sammlung von Mikroorganismen and Zellkulturen GmbH (DSMZ), Inhoffenstr. 7B, 38124 Braunschweig by Microbiotica Limited under an accession number as listed in Table 1b above and also shown below.

Thus, the therapeutic composition of the present invention may comprise at least one isolated bacterium, wherein the bacterium is a bacterium as deposited under the Budapest Treaty at DSMZ and assigned one of the following accession numbers (the date of deposit with DSMZ for each bacterium deposited is indicated in brackets after the accession number): DSM 33265 (13 Sep. 2019), DSM 33263 (13 Sep. 2019), DSM 33261 (13 Sep. 2019), DSM 33266 (13 Sep. 2019), DSM 33277 (13 Sep. 2019), DSM 33278 (13 Sep. 2019), DSM 33267 (13 Sep. 2019), DSM 33268 (13 Sep. 2019), DSM 33269 (13 Sep. 2019), DSM 33270 (13 Sep. 2019), DSM 33264 (13 Sep. 2019), DSM 33271 (13 Sep. 2019), DSM 33279 (13 Sep. 2019), DSM 33272 (13 Sep. 2019), DSM 33262 (13 Sep. 2019), DSM 33282 (13 Sep. 2019), DSM 33283 (13 Sep. 2019), DSM 33280 (13 Sep. 2019), DSM 33281 (13 Sep. 2019), DSM 33273 (13 Sep. 2019), DSM 33274 (13 Sep. 2019).

As explained above, the composition may comprise a combination of isolated strains, for example 2, 3, 4, 5, 6, 7, 8, 9, 10, 11, 12, 13, 14, 15, 16 or 17 of the isolated strains as listed above by accession number.

As will be apparent to a skilled person, different bacterial strains selected from the strains listed above can be combined in a single composition. For example, the composition comprises or consists of at least 2, e.g. up to 3, up to 4, up to 5, up to 6, up to 7, up to 8, up to 9, up to 10, up to 11, up to 12, up to 13, up to 14, up to 15, up to 16 or up to 17 isolated bacteria selected from those shown in table 1, for example with reference to the sequences as shown in the table. Typically, one bacterial strain is included for each species, but it is also possible to include more than 1, for example 2, 3, 4, 5, for each species.

In one embodiment, the composition comprises or consists of 4, 5, 7, 10 or 15 isolated bacterial strains listed by accession number above. In one embodiment, composition comprises or consists of the strains as shown for composition B, C and D (see examples).

In one embodiment, the composition comprises or consists of at least 2, at least 3, at least 4, at least 5, at least 6, at least 7, at least 8, at least 9, at least 10, at least 11, at least 12, at least 13, at least 14, at least 15, at least 16 or at least 17 isolated strains listed by accession number above.

In one embodiment, the composition comprises no more than 4, 7, 10 or 15 isolated strains listed by accession number above.

In one embodiment, the composition comprises or consists of 2 to 4, 2 to 5, 2 to 6, 2 to 7, 2 to 8, 2 to 9, 2 to 10, 2 to 11, 2 to 12, 2 to 13, 2 to 14, 2 to 15, 2 to 16 or 2 to 17 strains listed by accession number above.

In one embodiment, the composition comprises or consists of isolated bacterial strains selected from the following strains by reference to their accession number DSM 33265, DSM 33263, DSM 33261, DSM 33270, DSM 33264, DSM 33272 and/or DSM 33262. In one embodiment, the composition comprises or consists of isolated bacterial strains as described in the examples (see compositions A, B, C or D).

However, as explained above, the invention is not limited to specific strains, but encompasses bacteria defined by 16S rDNA. Thus, derivatives of a strain disclosed herein and deposited with the accession number as disclosed may be modified for example at the genetic level, without ablating the biological activity. In particular, a derivative strain of the invention is therapeutically active. A derivative strain will have comparable activity to the original deposited strain. In particular, a derivative strain will elicit comparable effects on C. difficile disease models and this may be identified by using the culturing and administration protocols described in the examples. A derivative of a deposited strain is of the same biotype. A biotype is a closely related strain that has the same or very similar physiological and biochemical characteristics. In certain embodiments strains that are biotypes of the bacteria deposited under the accession numbers listed herein and that are suitable for use in the invention are strains that provide the same pattern as the bacteria deposited under accession numbers listed herein when analysed by amplified ribosomal DNA restriction analysis (ARDRA), for example when using Sau3AI restriction enzyme analysis.

As explained herein, the bacterial compositions of the invention have therapeutic effects when administered to a subject and can be used in the treatment or prevention of C. difficile infection. In particular, the bacterial compositions of the invention have therapeutic effects when used in the C. difficile disease models described in the examples. Thus, the compositions as described here are pharmaceutical compositions.

In one embodiment, the composition may comprise a pharmaceutically acceptable excipient, carrier, buffer, stabilizer or other materials well known to those skilled in the art. Such materials should be non-toxic and should not interfere with the efficacy of the isolated bacteria present in the therapeutic composition. The precise nature of the pharmaceutically acceptable excipient or other material will depend on the route of administration, which may be oral or rectal. Many methods for the preparation of therapeutic compositions are known to those skilled in the art.

The bacterial compositions of the invention may comprise a prebiotic, a pharmaceutically acceptable carrier, insoluble fibre, a buffer, an osmotic agent, an anti-foaming agent and/or a preservative. Particular examples of excipients included in the composition are disclosed below.

Prebiotics may provide nutrients for the isolated bacteria present in the bacterial composition to assist their early growth and colonisation after administration to the individual. Any prebiotic known in the art may be used. Non-limiting examples of prebiotics include oligosaccharides, e.g., fructooligosaccharides such as oligofructose and inulin, mannan oligosaccharides and galactooligosaccharides, soluble, oligofructose-enriched inulin and soluble fibre. Insoluble fibre may be included in the therapeutic composition as a carrier, e.g., to provide protection during transit or storage. A buffer may be included in the bacterial composition to promote the viability of the isolated bacteria present. An anti-fungal agent may be included in the bacterial composition as a preservative.

In one embodiment, the therapeutic bacterial compositions may comprise no other active ingredient other than the bacterial isolates as described herein, including no other isolated bacterium, and optionally a prebiotic. Thus, the active ingredient of the therapeutic composition may consist of the group of bacterial isolates as described herein, and optionally a prebiotic.

The bacterial compositions of the invention can be administered to a subject in a variety of ways as described in more detail elsewhere herein, including in the form of a tablet, capsule, lozenge or liquid.

The bacterial compositions of the invention may be for oral or rectal administration to the subject. Where the composition is for oral administration, the composition may be in the form of a capsule, or a tablet. Where the therapeutic composition is for rectal administration, the therapeutic composition may be in the form of an enema. The preparation of suitable capsules, tablets and enema is well-known in the art. The capsule or tablet may comprise a coating to protect the capsule or tablet from stomach acid. For example, the capsule or tablet may be enteric-coated, pH dependant, slow-release, and/or gastro-resistant. Such capsules and tablets are used, for example, to minimize dissolution of the capsule or tablet in the stomach but allow dissolution in the small intestine. When intended for oral administration, the composition can be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like. Such a solid composition typically contains one or more inert diluents. In addition, one or more of the following can be present: binders such as carboxymethylcellulose, ethyl cellulose, microcrystalline cellulose, or gelatin, excipients such as starch, lactose or dextrins, disintegrating agents such as alginic acid, sodium alginate, corn starch and the like; lubricants such as magnesium stearate, glidants such as colloidal silicon dioxide, sweetening agents such as sucrose or saccharin, a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The bacterial composition may include a pharmaceutically acceptable carrier or vehicle can be particulate, so that the compositions are, for example, in tablet or powder form. The term “carrier” refers to a diluent, adjuvant or excipient, with which the composition is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, the composition and pharmaceutically acceptable carriers are sterile. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions can take the form of one or more dosage units. In an embodiment, the dose unit comprises at least 1×10³, 1×10⁴, 1×10⁵, 1×10⁶, 1×10⁷, 1×10⁸, 1×10⁹, 1×10¹⁰, 1×10¹¹ or greater than 1×10¹¹ colony forming units (cfu) of vegetative bacterial cells. In an embodiment, the dose unit comprises a pharmaceutically acceptable excipient, an enteric coating or a combination thereof. The bacterial isolates or composition may be provided at a dose of 1-100 g/day, such as 5, 10, 15, 20, 30, 40, 50, 60, 70, 80 or 90 g/day.

Treatments or specific processes can be applied to improve the stability or viability of the bacterial isolates in the composition. The bacterial composition can be applied in a dry form or in a wet from. The bacterial composition may be lyophilized. The lyophilized therapeutic composition may comprise one or more stabilisers and/or cryoprotectants. The lyophilized bacterial composition may be reconstituted using a suitable diluent prior to administration to the individual.

In another aspect, there is provided a method for treating or preventing a disease in a subject comprising administering a bacterial composition described herein. In another aspect, there is provided a bacterial composition described herein for use in the treatment of disease. In another aspect, there is provided the use of a bacterial composition described herein in the manufacture of a medicament for the treatment or prevention of a disease.

In one embodiment, the disease is an enteric pathogenic infection. The causative agents of enteric infections may be: C. difficile, Extended spectrum beta-lactamase producing Enterobacteriaceae, Third-generation cephalosporin-resistant Enterobacteriaceae, Carbapenem-resistant Enterobacteriaceae, Fluoroquinolone-resistant Enterobacteriaceae, Salmonella spp. including S. typhi, S. paratyphi, S. enteritidis, Escherichia coli including enteroinvasive E. coli, enterohemorrhagic E. coli, Shigella toxin-producing E. coli, Klebsiella pneumoniae, Vibrio Cholerae, Campylobacter spp. including Campylobacter jejeuni, C. coli, C. lari, C. fetus, Shigella spp. including S. dysenteriae, S. flexneri, S. boydii, S. sonnei, Cryptosporidium spp., Microsporidium spp., Entamoeba histolytica, Giardia lamblia, Blastocystis spp. including B. hominis.

In one embodiment, the disease is C. difficile infection.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., human patients, being treated. Many medical treatments are effective for some, but not all, patients that undergo the treatment.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

A bacterial composition according to the present invention may be administered alone or in combination with other treatments, concurrently or sequentially or as a combined preparation with another therapeutic agent or agents.

Administration may be in a “therapeutically effective amount”, this being sufficient to show benefit to the individual. Such benefit may be at least amelioration of at least one symptom. Thus “treatment” of a specified disease refers to amelioration of at least one symptom. The actual amount administered, and rate and time-course of administration, will depend on the nature and severity of what is being treated, the particular patient being treated, the clinical condition of the individual patient, the site of delivery of the composition, the type of therapeutic composition, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. A therapeutically effective amount or suitable dose of a therapeutic composition of the invention can be determined by comparing its in vitro activity and in vivo activity in an animal model. Methods for extrapolation of effective dosages in mice and other test animals to humans are known. The precise dose will depend upon a number of factors, including whether the therapeutic composition is for prevention or for treatment.

In one embodiment, the subject has not undergone treatment with an antibiotic prior to treatment with the composition described herein. In another embodiment, the subject has undergone treatment with an antibiotic prior to treatment with the composition described herein.

The methods and compositions described herein can be used in treating C. difficile infection or preventing C. difficile infection. For example, the method may reduce susceptibility to C. difficile infection. In one embodiment, the C. difficile infection is a first occurrence C. difficile infection. In one embodiment, the C. difficile infection is a recurrent C. difficile infection.

In another aspect, the invention relates to a method for increasing diversity of the gastrointestinal microbiota in a subject comprising administering a composition described herein.

In another aspect, the invention relates to a method of altering the microbiota of the gastrointestinal tract in a subject comprising administering a composition described herein.

In one embodiment of the methods which require administration of the composition, the method includes the further step of detecting the presence one or more of the bacterial strain that has been administered in the subject subsequent to administration. Methods for detection include for example detecting a 16S nucleic acid sequence as defined herein of at least one administered bacterial isolate in said subject.

The composition of the present invention may be prepared by a method comprising culturing the two or more isolated bacteria present in the composition in a suitable medium or media. Media and conditions suitable for culturing the bacteria to be included in the therapeutic composition of the present invention are described in detail elsewhere herein. For example, a method of preparing a therapeutic composition according to the present invention may comprise the steps of:

(i) culturing a first isolated bacterium as described herein;

(ii) culturing a second and optionally a further isolated bacterium; and

(iii) mixing the bacteria obtained in (i) and (ii) to prepare the therapeutic composition.

The isolated bacteria to be included in the composition may be cultured in separate steps. In other words, a separate culture of each bacterium to be included in the therapeutic composition is preferably prepared. This allows the growth of each bacterium to be evaluated and the amount of each bacterium to be included in the pharmaceutical composition to be controlled as desired. The bacteria cultured in steps (i) and (ii) preferably have distinct 16S nucleic acid sequences, that is 16S nucleic acid sequences that share less than 99%, 98%, 97%, 96% or 95% sequence identity.

The above method may include steps of culturing each isolated bacterium which is to be included in the composition.

The method may optionally comprise one or more further steps in which the bacteria are mixed with one or more additional ingredients, such as a pharmaceutically acceptable excipient, prebiotic, carrier, insoluble fibre, buffer, osmotic agent, antifoaming agent, and/or preservative. In addition, or alternatively, the method may comprise suspending the bacteria obtained in (i) and optionally (ii) in a chemostat medium, or saline, e.g. 0.9% saline. The bacteria obtained in (i) and optionally (ii) may be provided under a reduced atmosphere, such as N₂, CO₂, H₂, or a mixture thereof, e.g. N₂:CO₂:H₂. The gases may be present in appropriate ratios for the preservation of the bacteria present in the therapeutic composition. For example, the reduced atmosphere may comprise 80% N2, 10% CO₂ and 10% H₂. In addition, or alternatively, the method may comprise a step of lyophilising the bacteria obtained in (i) and optionally (ii), optionally in the presence of a stabiliser and/or cryprotectant. The method may also comprise a step of preparing a capsule, tablet, or enema comprising the bacteria obtained in (i) and optionally (ii). The capsule or tablet may be enteric-coated, pH dependant, slow-release, and/or gastro-resistant.

The composition of the invention may also be provided in the form of a food supplement to promote a healthy and balanced microbiome, for example as a beverage or other food stuff.

In a further aspect, the invention relates to a kit. The kit includes a composition described herein. In an example, the kit can include materials to ship the collected material without harming the samples (e.g., packaged in lyophilized form, or packaged in an aqueous medium etc.). The kit may include the processed material or treatment in a sterile container, such as a nasogastric (NG) tube, a vial (e.g., for use with a retention enema), a gastro-resistant capsule (e.g., acid-bio resistant to reach the intestinal tract, having a sterile outside), etc. The kit may also comprise instructions for use.

In another aspect, the invention relates to a use of one or more bacteria selected from Table 1 in a method for identifying a donor for FMT therapy.

In another aspect, the invention relates to a method for treating a faecal transplant prior to administration to a subject comprising supplementing the faecal transplant with one or more isolated bacteria selected from Table 1a or b.

Further aspects and embodiments of the invention will be apparent to those skilled in the art given the present disclosure including the following experimental exemplification.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including any references to gene accession numbers and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The word “substantially” does not exclude “completely” e.g. a composition which is “substantially free” from Y may be completely free from Y.

The invention is further described in the non-limiting examples.

### EXAMPLES

### Example 1 Identification and Isolation of Bacterial Isolates

An experiment was performed to screen the gut microbiota of healthy human donors for the ability to prevent C. difficile infection in germ-free mice. Experiments were performed using C57BL/6 germ-free mice inoculated with faecal slurries prepared from healthy donor stool. Mice were administered C. difficile M7404 (serotype 027) by gavage. To generate mice with a humanised microbiota, FMT using human stool (equivalent of 20 mg stool per mouse) was performed once a week for three consecutive weeks followed by a “settling in” period. Following the introduction of C. difficile, stool samples were collected frequently between day 1 and day 28 for culture-based enumeration of C. difficile load and for evaluation of microbiota composition by shotgun metagenomics. Stool samples were collected prior to any interventions that were scheduled, such as gavage.

A microbiota was considered protective against C. difficile disease if there was no evidence of C. difficile shedding from any of the mice in a single cage at every timepoint up to and including day 21. The C. difficile load borne by each mouse was enumerated by selective plating of stool homogenate on CCEY agar followed by anaerobic incubation at 37° C.

Several C. difficile shedding phenotypes emerged, depending on the donor. One donor completely prevented C. difficile shedding out to day 21. This cohort was used for further analysis to identify therapeutically beneficial bacteria.

The microbiota profile of the mice in each cage at day 0 prior to inoculation with C. difficile spores was hypothesised to be predictive of protection against C. difficile shedding. Differences between bacteria in the donors were investigated. Thus, shotgun metagenomics was performed on all stool samples collected at D0. The metagenomics data were analysed with a machine learning approach. This approach integrated microbiome data and the C. difficile shedding phenotype of the mice to identify a list of bacteria that are likely to contribute to the protective phenotype.

This yielded a list of 15 different bacterial strains that were likely to confer a protective effect.

A culture collection of commensal bacteria from one human donor was generated to provide a resource from which a bacteriotherapy consortium could be made. The principal advantage of obtaining the candidate therapeutic bacteria from a single healthy human donor is that these bacteria naturally occur together and so are likely to be compatible with each other. Moreover, they are all derived from a microbiota that is known to be protective against C. difficile carriage, providing a basis for their therapeutic function.

Strains were chosen for inclusion in the bacteriotherapy mix based on their 16S rRNA gene matching the 16SrRNA gene of the isolates identified as having a therapeutic effect by the machine learning approaches described above.

Two strains (Bifidobacterium longum and Bacteroides sp), that were also recovered from the donor whose microbiota completely prevented C. difficile shedding out to day 21, were additionally included in the therapeutic composition as primary colonisers. The rationale for their inclusion was to improve colonisation by candidate therapeutic isolates that may be secondary and tertiary colonisers of the gut. The consortium of 17 isolated bacteria, and subsets, was subsequently tested in in vivo assays.

### Example 2 In Vivo Assays

1) Testing of a Composition of 17 Bacterial Isolates and Subsets of the 17 Bacterial Isolates in Vivo in a Survival Model of C. difficile Infection

A composition having 17 isolated bacteria (composition A) as set out in table 1 was tested in an animal model. 16S rDNA sequence IDs of the isolates are provided in table 1. For Bacteroides cellulosilyticus, SEQ ID NO. 1 (DSM 33265) was used. For Parabacteroides distasonis, SEQ ID NO. 10 (DSM 33270) was used. For Bacteroides fragilis, SEQ ID NO. 14 (DSM 33272) was used.

A murine C. difficile infection model was established, as previously described (14). The model is based upon suppression of the resident mouse microbiota using antibiotics, creating a nutritional niche for C. difficile to grow, leading to the development of disease.

Female specific pathogen free (SPF) C57/BL6N mice approximately 6 weeks old were used throughout. The mice were pre-conditioned for 10 days by treatment with cefoperazone prepared in sterile drinking water (0.5 mg/mL, starting on day minus 12 until day minus 2 relative to infection). Mice were allowed to drink the antibiotic containing water ad libitum. Mice were returned to drinking water free of cefoperazone 48h prior to infection for the remaining duration of the study. 48h prior to infection with C. difficile, a single dose of clindamycin (10 mg/kg) was administered by intraperitoneal injection.

A day prior to infection with C. difficile, mice were treated with two doses of Composition A at 1-2×10⁷ cfu administered 11 hours apart. On day 0, the mice were infected with C. difficile by administration of 100 μl of spore preparation from C. difficile strain ATCC 43255 (VPI 10463), corresponding to 10⁶ cfu of spores.

Following infection, mice were monitored as frequently as clinically required for signs of C. difficile infection with the aim that no mouse suffered prolonged severe disease or death. Signs of infection include pyrexia, loose stools and weight loss. Mice found with severe wet tail, diarrhoea, hypothermia, lying prone or unresponsive were euthanized and the time of death together with clinical condition recorded.

Survival results are shown in FIG. 1. The efficacy of composition A was tested in comparison to treatment with FMT therapy and the vehicle alone. A robust model of C. difficile ATCC 43255 infection was established with vehicle mice showing a mean survival time of 34 hours post infection in the vehicle treatment group. Mice treated with the Composition A and FMT showed a mean survival of ˜122 and ˜121 hours respectively. Both of these treatment groups showed superior survival compared to vehicle treated mice, and this was statistically significant (Log Rank test, p=0.0008). This demonstrates that the composition is efficacious and increases survival rates.

A composition having 15 isolated bacteria (composition B) as set out in table 2 was tested in the survival model. 16S rDNA sequence ID Nos of the isolates are provided in table 2. Mice were treated with two doses of Composition B at 0.6-1×10⁷ cfu administered 11 hours apart.

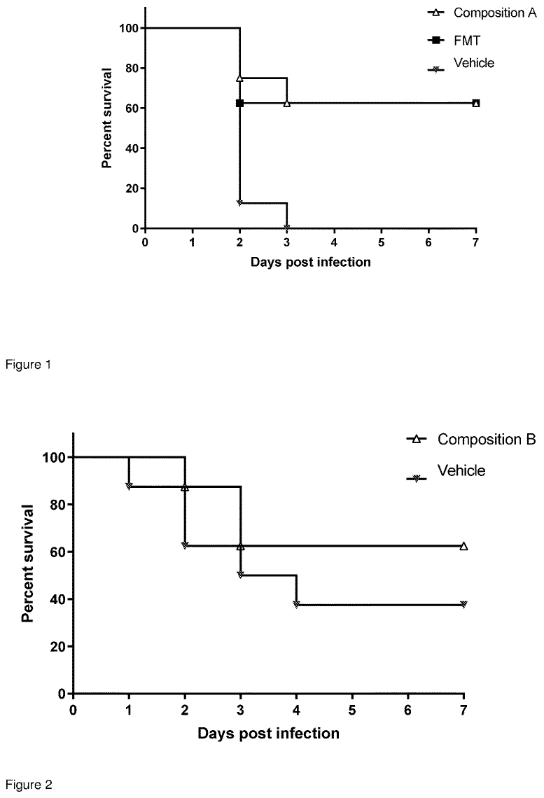

The efficacy of Composition B was tested in comparison to treatment with the vehicle, assessing survival over 7 days (FIG. 2). A robust model of C. difficile ATCC 43255 infection was established with vehicle mice showing a mean survival time of 91 hours post infection in the vehicle treatment group. Mice treated with Composition B showed a mean survival of ˜128 hours. Seven days post infection, survival was 38% in the vehicle treated mice compared to 63% in the Composition B treated mice. This demonstrates that composition B is efficacious and increases survival rates.

TABLE 2
Bacterial strains in Composition B.

| | | 16S rDNA | |
|---|---|---|---|
| | | sequence - | DSMZ |
| | | sequence | Accession |
| No | Taxonomy | identifier | Number |
| 1 | Bacteroides cellulosilyticus | SEQ ID NO. 1 | DSM 33265 |
| 2 | Blautia sp. | SEQ ID NO. 4 | DSM 33266 |
| 3 | Coprococcus catus | SEQ ID NO. 5 | DSM 33277 |
| 4 | Coprococcus comes | SEQ ID NO. 6 | DSM 33278 |
| 5 | Dorea sp. | SEQ ID NO. 7 | DSM 33267 |
| 6 | Erysipelotrichaceae UCG-003 | SEQ ID NO. 8 | DSM 33268 |
| | sp. | | |
| 7 | Odoribacter splanchnicus | SEQ ID NO. 9 | DSM 33269 |
| 8 | Parabacteroides distasonis | SEQ ID NO. 10 | DSM 33270 |
| 9 | Ruminiclostridium 9 sp. | SEQ ID NO. 12 | DSM 33271 |
| 10 | Ruminococcus torques | SEQ ID NO. 13 | DSM 33279 |
| 11 | Bacteroides fragilis | SEQ ID NO. 14 | DSM 33272 |
| 12 | Blautia sp. | SEQ ID NO. 16 | DSM 33282 |
| 13 | Blautia sp. | SEQ ID NO. 17 | DSM 33283 |
| 14 | Lachnospiraceae FCS020 | SEQ ID NO. 18 | DSM 33280 |
| | group sp. | | |
| 15 | Lachnoclostridium sp. | SEQ ID NO. 19 | DSM 33281 |

A composition having 10 isolated bacteria (composition C) as set out in table 3 was tested in the survival model. 16S rDNA sequence ID Nos of the isolates are provided in table 3. Mice were treated with two doses of Composition C at 0.9-2×10⁸ cfu administered 11 hours apart on Day −1 and 1 (mice infected with C. difficile spores on day 0). Mice were given a single dose of Composition C at of 0.7×10⁸ cfu on day 0, 11 hours prior to infection with C. difficile.

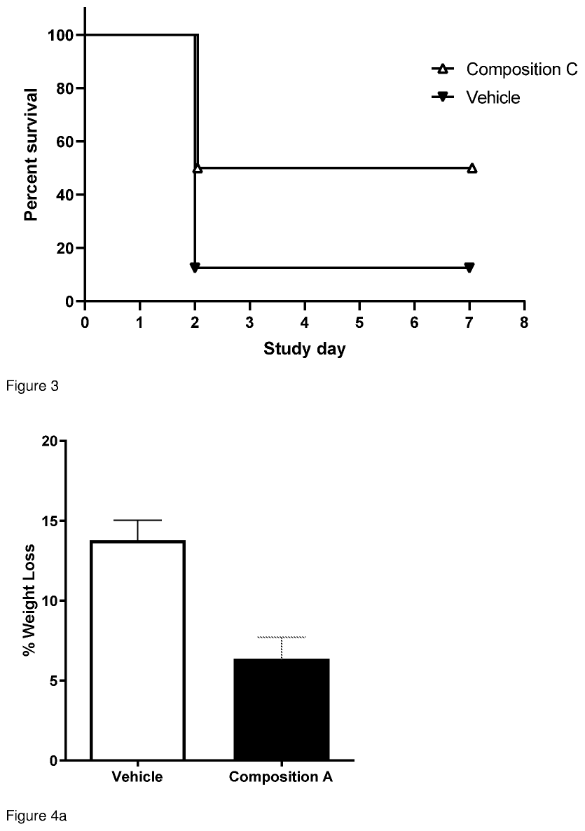

The efficacy of Composition C was tested in comparison to treatment with the vehicle, assessing survival over 7 days (FIG. 3). A robust model of C. difficile ATCC 43255 infection was established with mice showing a mean survival time of 55 hours post infection in the vehicle treatment group. Mice treated with Composition C showed a mean survival of 107 hours. Seven days post infection, survival was 13% in the vehicle treated mice compared to 50% in the Composition C treated mice. This demonstrates that composition C is efficacious and increases survival rates.

TABLE 3
Bacterial strains in Composition C.

| | | 16S rDNA | |
|---|---|---|---|
| | | sequence - | DSMZ |
| | | sequence | Accession |
| No | Taxonomy | identifier | Number |
| 1 | Bacteroides cellulosilyticus | SEQ ID NO. 1 | DSM 33265 |
| 2 | Blautia sp. | SEQ ID NO. 4 | DSM 33266 |
| 3 | Coprococcus catus | SEQ ID NO. 5 | DSM 33277 |
| 4 | Coprococcus comes | SEQ ID NO. 6 | DSM 33278 |
| 5 | Dorea sp. | SEQ ID NO. 7 | DSM 33267 |
| 6 | Erysipelotrichaceae UCG- | SEQ ID NO. 8 | DSM 33268 |
| | 003 sp. | | |
| 7 | Odoribacter splanchnicus | SEQ ID NO. 9 | DSM 33269 |
| 8 | Parabacteroides distasonis | SEQ ID NO. 10 | DSM 33270 |
| 9 | Ruminiclostridium 9 sp. | SEQ ID NO. 12 | DSM 33271 |
| 10 | Ruminococcus torques | SEQ ID NO. 13 | DSM 33279 |

2) Testing of the 17 Bacterial Isolates and Subsets of the 17 Bacterial Isolates In Vivo, in a Weight Loss Model of C. difficile Infection

In further experiments, composition A and two other bacterial compositions, composition C and composition D comprising 10 and 4 strain subsets of the isolates shown in table 1 respectively, were tested in an animal model of C. difficile infection. The bacterial isolates in compositions C and D are shown in Table 3 and 4 respectively, with the respective 16S rDNA sequences.

TABLE 4
Bacterial strains in composition D.

| | | DSMZ |
|---|---|---|
| | 16S rDNA sequence - | Accession |
| Taxonomy | sequence identifier | Number |
| Bacteroides cellulosilyticus | SEQ ID NO. 3 | DSM 33261 |
| Parabacteroides distasonis | SEQ ID NO. 11 | DSM 33264 |
| Bacteroides fragilis | SEQ ID NO. 15 | DSM 33262 |
| Bacteroides sp. | SEQ ID NO. 21 | DSM 33274 |

A murine C. difficile infection model was established based upon a model previously described (14). The model is based upon suppression of the resident mouse microbiota using a cocktail of antibiotics, creating a nutritional niche for C. difficile to grow, leading to the development of disease.

Female SPF mice (strain C57BL/6) between 5-9 weeks old were used. Mice were given water (ad libitum) supplemented with kanamycin (0.4 mg/mL), gentamicin (0.035 mg/mL), colistin (850 U/mL), metronidazole (0.215 mg/mL), and vancomycin (0.045 mg/mL), for three days (days −7 to −4), then switched to antibiotic-free water (ad libitum). On day −2 the mice were given a single dose of clindamycin (10 mg/kg) by oral gavage.

Mice were treated with LBP or vehicle on day −1, or on day −1 and day 0, by oral gavage. On day 0 each mouse was challenged with 1×10⁵ CFU of spores of C. difficile strain M7404 (ribotype 027). Mice were monitored for weight loss, as a sign of disease.

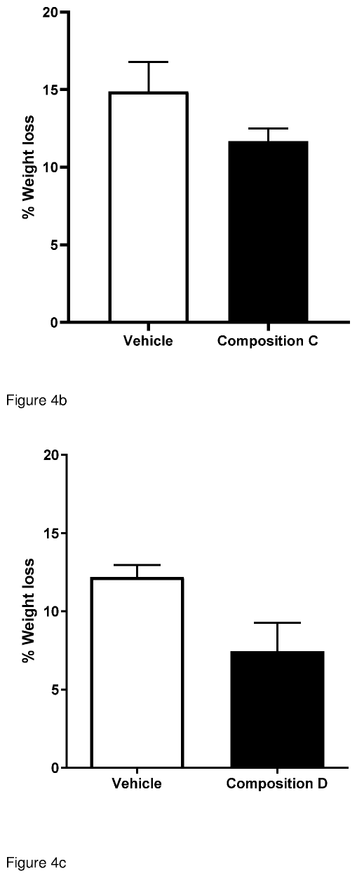

A single dose of the 17 strain consortium, Composition A, comprising 6-8×10⁷ cfu administered one day prior to C. difficile infection provided protection from weight loss in the mice, compared to the vehicle control—two days post C. difficile challenge mice in the Composition A-treated group had lost an average of 6.4% of their body weight, compared to a loss of 13.8% in the vehicle-treated group (FIG. 4a), which was statistically significant (t-test, p=<0.006). This increased protection from C. difficile infection-related weight loss, compared to the vehicle-treated mice, shows that composition A was efficacious.

The 10 strain consortium (Composition C) was tested in the same model, administered to the mice one day prior to infection and on the day of infection, five hours prior to infection, dosing at 1-2×10⁷ cfu on both occasions. In comparison to vehicle treatment, composition C was protective against weight loss caused by C. difficile infection—two days post C. difficile challenge mice in the composition C-treated group had lost an average of 11.7% of their body weight, compared to a loss of 14.9% in the vehicle-treated group (FIG. 4b). This increased protection from C. difficile infection-related weight loss, compared to the vehicle-treated mice, shows that composition C was efficacious.

Faecal samples were isolated from mice treated with Composition A, and total DNA was extracted and purified from the faecal samples. The faecal DNA samples were subjected to PCR, using 17 primer pairs in separate reactions, each pair being specific to one of the strains in the 17 strains of composition A. PCR amplification products were obtained from faecal samples obtained 3 or more days post administration, for 4 of the primer sets specific to B. ceullosilyticus, P. distasonis, B. fragilis, Bacteroies sp. from the 17 strains listed in table 1. It was considered that these particular strains may be able to prevent the outgrowth of C. difficile in the gastrointestinal tract. These 4 strains were formulated into composition D, and this composition was tested in the weight loss model of C. difficile infection, described above. Composition D was administered twice one day prior to C. difficile infection, with two doses of 5×10⁷ cfu administered 8 hours apart. In comparison to vehicle treatment, composition D was protective against weight loss caused by C. difficile infection—two days post C. difficile challenge mice in the Composition D-treated group had lost an average of 7.4% of their body weight, compared to a loss of 12.2% in the vehicle-treated group (FIG. 4c), which was statistically significant (t-test, p=0.03). This increased protection from C. difficile infection-related weight loss, compared to the vehicle-treated mice, shows that composition D was efficacious.

### REFERENCES

1. Leffler D A, Lamont J T. Clostridium difficile infection. N Engl J Med. 2015; 372(16)1539-48.
2. Speight S, Moy A, Macken S, Chitnis R, Hoffman P N, Davies A, et al. Evaluation of the sporicidal activity of different chemical disinfectants used in hospitals against Clostridium difficile. J Hosp Infection. 2011; 79(1):18-22.
3. Control Cf D. Antibiotic Resistance Threats in the United States, 2013. 2013.
4. Lessa F C, Mu Y, Bamberg W M, Beldays Z G, Dumyati G K, Dunn J R, et al. Burden of Clostridium difficile infection in the United States. N Engl J Med. 2015; 372(9):825-34.
5. Control ECfDPa. Annual Epidemiological Report for 2016: Healthcare-associated infections: Clostridium difficile infections. Stockholm; 218 June 2018.
6. Nelson R L, Suda K J, Evans C T. Antibiotic treatment for Clostridium difficile-associated diarrhoea in adults. Cochrane Database Syst Rev. 2017; 3:CD004610.
7. Cammarota G, Ianiro G, Tilg H, Rajilic-Stojanovic M, Kump P, Satokari R, et al. European consensus conference on faecal microbiota transplantation in clinical practice. Gut. 2017; 66(4):569-80.
8. Mamo Y, Woodworth M H, Wang T, Dhere T, Kraft C S. Durability and Long-term Clinical Outcomes of Fecal Microbiota Transplant Treatment in Patients With Recurrent Clostridium difficile Infection. Clin Infect Dis. 2018; 66(11):1705-11.
9. Sachs R E, Edelstein C A. Ensuring the safe and effective FDA regulation of fecal microbiota transplantation. J Law Biosci. 2015; 2(2):396-415.
10. Orenstein R, Dubberke E, Hardi R, Ray A, Mullane K, Pardi D S, et al. Safety and Durability of RBX2660 (Microbiota Suspension) for Recurrent Clostridium difficile Infection: Results of the PUNCH C D Study. Clin Infect Dis. 2016; 62(5):596-602.
11. Khanna S, Pardi D S, Kelly C R, Kraft C S, Dhere T, Henn M R, et al. A Novel Microbiome Therapeutic Increases Gut Microbial Diversity and Prevents Recurrent Clostridium difficile Infection. J Infect Dis. 2016; 214(2):173-81.
12. U. S. Department of Health and Human Services FaDA. Early Clinical Trials with Live Biotherapeutic Products: Chemistry, Manufacturing, and Control Information: Guidance for industry. 216 February 2012.
13. Lawley T D, Clare S, Walker A W, Stares M D, Connor T R, Raisen C, et al. Targeted restoration of the intestinal microbiota with a simple, defined bacteriotherapy resolves relapsing Clostridium difficile disease in mice. PLoS Pathog. 2012; 8(10):e1002995.
14. Warn, P., Thommes, P., Sattar, A., Corbett, D., Flattery, A., Zhang, Z., Black, T., Hernandez, L. D. and Therien, A. G., 2016. Disease progression and resolution in rodent models of Clostridium difficile infection and impact of antitoxin antibodies and vancomycin. Antimicrobial agents and chemotherapy, 60(11), pp. 6471-6482.
15. Chen, X., Katchar, K., Goldsmith, J. D., Nanthakumar, N., Cheknis, A., Gerding, D. N. and Kelly, C. P., 2008. A mouse model of Clostridium difficile-associated disease. Gastroenterology, 135(6), pp. 1984-1992.

### SEQUENCES

>Bacteroides cellulosilyticus
SEQ ID NO. 1
AGAGTTTGATCCTGGCTCAGGATGAACGCTAGCTACAGGCTTAACACATGCAAGTCGAGG
GGCAGCATGACCTAGCAATAGGTTGATGGCGACCGGCGCACGGGTGAGTAACACGTATCC
AACCTACCGGTTATTCCGGGATAGCCTTTCGAAAGAAAGATTAATACCGGATAGTATAAC
GAGAAGGCATCTTTTTGTTATTAAAGAACTTCGATAACCGATGGGGATGCGTTCCATTAG
TTTGTTGGCGGGGTAACGGCCCACCAAGACATCGATGGATAGGGGTTCTGAGAGGAAGGT
CCCCCACATTGGAACTGAGACACGGTCCAAACTCCTACGGGAGGCAGCAGTGAGGAATAT
TGGTCAATGGACGAGAGTCTGAACCAGCCAAGTAGCGTGAAGGATGACTGCCCTATGGGT
TGTAAACTTCTTTTATATGGGAATAAAGTGAGCCACGTGTGGCTTTTTGTATGTACCATA
CGAATAAGGATCGGCTAACTCCGTGCCAGCAGCCGCGGTAATACGGAGGATCCGAGCGTT
ATCCGGATTTATTGGGTTTAAAGGGAGCGTAGGCGGACTATTAAGTCAGCTGTGAAAGTT
TGCGGCTCAACCGTAAAATTGCAGTTGATACTGGTCGTCTTGAGTGCAGTAGAGGTAGGC
GGAATTCGTGGTGTAGCGGTGAAATGCTTAGATATCACGAAGAACTCCGATTGCGAAGGC
AGCTTACTGGACTGTAACTGACGCTGATGCTCGAAAGTGTGGGTATCAAACAGGATTAGA
TACCCTGGTAGTCCACACAGTAAACGATGAATACTCGCTGTTTGCGATATACAGCAAGCG
GCCAAGCGAAAGCATTAAGTATTCCACCTGGGGAGTACGCCGGCAACGGTGAAACTCAAA
GGAATTGACGGGGGCCCGCACAAGCGGAGGAACATGTGGTTTAATTCGATGATACGCGAG
GAACCTTACCCGGGCTTAAATTGCAAATGAATATAGTGGAAACATTATAGCCGCAAGGCA
TTTGTGAAGGTGCTGCATGGTTGTCGTCAGCTCGTGCCGTGAGGTGTCGGCTTAAGTGCC
ATAACGAGCGCAACCCTTATCTTTAGTTACTAACAGGTCATGCTGAGGACTCTAGAGAGA
CTGCCGTCGTAAGATGTGAGGAAGGTGGGGATGACGTCAAATCAGCACGGCCCTTACGTC
CGGGGCTACACACGTGTTACAATGGGGGGTACAGAAGGCAGCTACACAGCGATGTGATGC
TAATCCCAAAAGCCTCTCTCAGTTCGGATTGGAGTCTGCAACCCGACTCCATGAAGCTGG
ATTCGCTAGTAATCGCGCATCAGCCACGGCGCGGTGAATACGTTCCCGGGCCTTGTACAC
ACCGCCCGTCAAGCCATGAAAGCCGGGGGTACCTGAAGTCCGTAACCGCAAGGAGCGGCC
TAGGGTAAAACTGGTAATTGGGGCTAAGTCGTAACAAGGTAGCCGTACCGGAAGGTGCGG
CTG
>Bacteroides cellulosilyticus
SEQ ID NO. 2
AGAGTTTGATCCTGGCTCAGGATGAACGCTAGCTACAGGCTTAACACATGCAAGTCGAGG
GGCAGCATGACCTAGCAATAGGTTGATGGCGACCGGCGCACGGGTGAGTAACACGTATCC
AACCTACCGGTTATTCCGGGATAGCCTTTCGAAAGAAAGATTAATACCGGATAGTATAAC
GAGAAGGCATCTTCTTGTTATTAAAGAATTTCGATAACCGATGGGGATGCGTTCCATTAG
TTTGTTGGCGGGGTAACGGCCCACCAAGACATCGATGGATAGGGGTTCTGAGAGGAAGGT
CCCCCACATTGGAACTGAGACACGGTCCAAACTCCTACGGGAGGCAGCAGTGAGGAATAT
TGGTCAATGGACGAGAGTCTGAACCAGCCAAGTAGCGTGAAGGATGACTGCCCTATGGGT
TGTAAACTTCTTTTATATGGGAATAAAGTGAGCCACGTGTGGCTTTTTGTATGTACCATA
CGAATAAGGATCGGCTAACTCCGTGCCAGCAGCCGCGGTAATACGGAGGATCCGAGCGTT
ATCCGGATTTATTGGGTTTAAAGGGAGCGTAGGCGGACTATTAAGTCAGCTGTGAAAGTT
TGCGGCTCAACCGTAAAATTGCAGTTGATACTGGTCGTCTTGAGTGCAGTAGAGGTAGGC
GGAATTCGTGGTGTAGCGGTGAAATGCTTAGATATCACGAAGAACTCCGATTGCGAAGGC
AGCTTACTGGACTGTAACTGACGCTGATGCTCGAAAGTGTGGGTATCAAACAGGATTAGA
TACCCTGGTAGTCCACACAGTAAACGATGAATACTCGCTGTTTGCGATATACAGCAAGCG
GCCAAGCGAAAGCATTAAGTATTCCACCTGGGGAGTACGCCGGCAACGGTGAAACTCAAA
GGAATTGACGGGGGCCCGCACAAGCGGAGGAACATGTGGTTTAATTCGATGATACGCGAG
GAACCTTACCCGGGCTTAAATTGCAAATGAATATAGTGGAAACATTATAGCCGCAAGGCA
TTTGTGAAGGTGCTGCATGGTTGTCGTCAGCTCGTGCCGTGAGGTGTCGGCTTAAGTGCC
ATAACGAGCGCAACCCTTATCTTTAGTTACTAACAGGTCATGCTGAGGACTCTAGAGAGA
CTGCCGTCGTAAGATGTGAGGAAGGTGGGGATGACGTCAAATCAGCACGGCCCTTACGTC
CGGGGCTACACACGTGTTACAATGGGGGGTACAGAAGGCAGCTACACAGCGATGTGATGC
TAATCCCAAAAGCCTCTCTCAGTTCGGATTGGAGTCTGCAACCCGACTCCATGAAGCTGG
ATTCGCTAGTAATCGCGCATCAGCCACGGCGCGGTGAATACGTTCCCGGGCCTTGTACAC
ACCGCCCGTCAAGCCATGAAAGCCGGGGGTACCTGAAGTCCGTAACCGCAAGGAGCGGCC
TAGGGTAAAACTGGTAATTGGGGCTAAGTCGTAACAAGGTAGCCGTACCGGAAGGTGCGG
CTG
>Bacteroides cellulosilyticus
SEQ ID NO. 3
AGAGTTTGATCCTGGCTCAGGATGAACGCTAGCTACAGGCTTAACACATGCAAGTCGAGG
GGCAGCATGACCTAGCAATAGGTTGATGGCGACCGGCGCACGGGTGAGTAACACGTATCC
AACCTACCGGTTATTCCGGGATAGCCTTTCGAAAGAAAGATTAATACCGGATAGTATAAC
GAGAAGGCATCTTTTTGTTATTAAAGAATTTCGATAACCGATGGGGATGCGTTCCATTAG
TTTGTTGGCGGGGTAACGGCCCACCAAGACATCGATGGATAGGGGTTCTGAGAGGAAGGT
CCCCCACATTGGAACTGAGACACGGTCCAAACTCCTACGGGAGGCAGCAGTGAGGAATAT
TGGTCAATGGACGAGAGTCTGAACCAGCCAAGTAGCGTGAAGGATGACTGCCCTATGGGT
TGTAAACTTCTTTTATATGGGAATAAAGTGAGCCACGTGTGGCTTTTTGTATGTACCATA
CGAATAAGGATCGGCTAACTCCGTGCCAGCAGCCGCGGTAATACGGAGGATCCGAGCGTT
ATCCGGATTTATTGGGTTTAAAGGGAGCGTAGGCGGACTATTAAGTCAGCTGTGAAAGTT
TGCGGCTCAACCGTAAAATTGCAGTTGATACTGGTCGTCTTGAGTGCAGTAGAGGTAGGC
GGAATTCGTGGTGTAGCGGTGAAATGCTTAGATATCACGAAGAACTCCGATTGCGAAGGC
AGCTTACTGGACTGTAACTGACGCTGATGCTCGAAAGTGTGGGTATCAAACAGGATTAGA
TACCCTGGTAGTCCACACAGTAAACGATGAATACTCGCTGTTTGCGATATACAGCAAGCG
GCCAAGCGAAAGCATTAAGTATTCCACCTGGGGAGTACGCCGGCAACGGTGAAACTCAAA
GGAATTGACGGGGGCCCGCACAAGCGGAGGAACATGTGGTTTAATTCGATGATACGCGAG
GAACCTTACCCGGGCTTAAATTGCATCTGAATAATTTGGAAACAGATTAGCCGTAAGGCA
GATGTGAAGGTGCTGCATGGTTGTCGTCAGCTCGTGCCGTGAGGTGTCGGCTTAAGTGCC
ATAACGAGCGCAACCCTTATCTTTAGTTACTAACAGGTCATGCTGAGGACTCTAGAGAGA
CTGCCGTCGTAAGATGTGAGGAAGGTGGGGATGACGTCAAATCAGCACGGCCCTTACGTC
CGGGGCTACACACGTGTTACAATGGGGGGTACAGAAGGCAGCTACACAGCGATGTGATGC
TAATCCCAAAAGCCTCTCTCAGTTCGGATTGGAGTCTGCAACCCGACTCCATGAAGCTGG
ATTCGCTAGTAATCGCGCATCAGCCACGGCGCGGTGAATACGTTCCCGGGCCTTGTACAC
ACCGCCCGTCAAGCCATGAAAGCCGGGGGTACCTGAAGTCCGTAACCGTAAGGAGCGGCC
TAGGGTAAAACTGGTAATTGGGGCTAAGTCGTAACAAGGTAGCCGTACCGGAAGGTGCGG
CTG
>Blautia sp.
SEQ ID NO. 4
AGAGTTTGATCCTGGCTCAGGATGAACGCTGGCGGCGTGCTTAACACATGCAAGTCGAAC
GGGAAATACTTTATTGAAACTTCGGTGGATTTAATTTATTTCTAGTGGCGGACGGGTGAG
TAACGCGTGGGTAACCTGCCTTATACTGGGGGATAACAGCCAGAAATGACTGCTAATACC
GCATAAGCGCACAGAACCGCATGGTTCGGTGTGAAAAACTCCGGTGGTATAAGATGGACC
CGCGTTGGATTAGCTAGTTGGCAGGGCAGCGGCCTACCAAGGCGACGATCCATAGCCGGC
CTGAGAGGGTGAACGGCCACATTGGGACTGAGACACGGCCCAGACTCCTACGGGAGGCAG
CAGTGGGGAATATTGCACAATGGGGGAAACCCTGATGCAGCGACGCCGCGTGAAGGAAGA
AGTATCTCGGTATGTAAACTTCTATCAGCAGGGAAGATAATGACGGTACCTGACTAAGAA
GCCCCGGCTAACTACGTGCCAGCAGCCGCGGTAATACGTAGGGGGCAAGCGTTATCCGGA
TTTACTGGGTGTAAAGGGAGCGTAGACGGCGCAGCAAGTCTGATGTGAAAGGCAGGGGCT
TAACCCCTGGACTGCATTGGAAACTGCTGTGCTTGAGTGCCGGAGGGGTAAGCGGAATTC
CTAGTGTAGCGGTGAAATGCGTAGATATTAGGAGGAACACCAGTGGCGAAGGCGGCTTAC
TGGACGGTAACTGACGTTGAGGCTCGAAAGCGTGGGGAGCAAACAGGATTAGATACCCTG
GTAGTCCACGCCGTAAACGATGAATACTAGGTGTCAGGGAGCACAGCTCTTTGGTGCCGC
CGCAAACGCATTAAGTATTCCACCTGGGGAGTACGTTCGCAAGAATGAAACTCAAAGGAA
TTGACGGGGACCCGCACAAGCGGTGGAGCATGTGGTTTAATTCGAAGCAACGCGAAGAAC
CTTACCAAATCTTGACATCCCTCTGACCGGGACTTAACCGTCCCTTTCCTTCGGGACAGG
GGAGACAGGTGGTGCATGGTTGTCGTCAGCTCGTGTCGTGAGATGTTGGGTTAAGTCCCG
CAACGAGCGCAACCCCTATCCTTAGTAGCCAGCACGTAATGGTGGGCACTCTGAGGAGAC
TGCCAGGGATAACCTGGAGGAAGGCGGGGATGACGTCAAATCATCATGCCCCTTATGATT
TGGGCTACACACGTGCTACAATGGCGTAAACAAAGGGAAGCGAACCCGCGAGGGTGGGCA
AATCTCAAAAATAACGTCCCAGTTCGGACTGCAGTCTGCAACTCGACTGCACGAAGCTGG
AATCGCTAGTAATCGCGGATCAGAATGCCGCGGTGAATACGTTCCCGGGTCTTGTACACA
CCGCCCGTCACACCATGGGAGTCAGTAACGCCCGAAGTCAGTGACCTAACCGCAAGGGAG
GAGCTGCCGAAGGCGGGACCGATGACTGGGGTGAAGTCGTAACAAGGTAGCCGTATCGGA
AGGTGCGGCTG
>Coprococcus catus
SEQ ID NO. 5
GTTTGATCCTGGCTCAGGATGAACGCTGGCGGCGTGCTTAACACATGCAAGTCGAACGGA
CGATGAAGAGCTTGCTTTTCAGAGTTAGTGGCGGACGGGTGAGTAACGCGTGGGTAACCT
GCCTCATACAGGGGGATAGCAGCTGGAAACGGCTGATAAAACCGCATAAGCGCACAGCAT
CGCATGATGCAGTGTGAAAAACTCCGGTGGTATGAGATGGACCCGCGTCTGATTAGCTGG
TTGGTGAGGTAACGGCCCACCAAGGCGACGATCAGTAGCCGGCCTGAGAGGGTGACCGGC
CACATTGGGACTGAGACACGGCCCAAACTCCTACGGGAGGCAGCAGTGGGGAATATTGCA
CAATGGGGGAAACCCTGATGCAGCGACGCCGCGTGAAGGAAGAAGTATCTCGGTATGTAA
ACTTCTATCAGCAGGGAAGATAATGACGGTACCTGACTAAGAAGCCCCGGCTAACTACGT
GCCAGCAGCCGCGGTAATACGTAGGGGGCAAGCGTTATCCGGATTTACTGGGTGTAAAGG
GAGCGTAGGCGGCGGAGCAAGTCAGAAGTGAAAGCCCGGGGCTCAACCCCGGGACGGCTT
TTGAAACTGCCCTGCTTGATTTCAGGAGAGGTAAGCGGAATTCCTAGTGTAGCGGTGAAA
TGCGTAGATATTAGGAGGAACACCAGTGGCGAAGGCGGCTTACTGGACTGACAATGACGC
TGAGGCTCGAAAGCGTGGGGAGCAAACAGGATTAGATACCCTGGTAGTCCACGCCGTAAA
CGATGAATACTAGGTGTCGGGGGCCATAAGGCTTTCGGTGCCGCAGCAAACGCAATAAGT
ATTCCACCTGGGGAGTACGTTCGCAAGAATGAAACTCAAAGGAATTGACGGGGACCCGCA
CAAGCGGTGGAGCATGTGGTTTAATTCGAAGCAACGCGAAGAACCTTACCAGGCCTTGAC
ATCCCGGTGACCGTCCCGTAATGGGGACCTCTCTTCGGAGCACCGGTGACAGGTGGTGCA
TGGTTGTCGTCAGCTCGTGTCGTGAGATGTTGGGTTAAGTCCCGCAACGAGCGCAACCCC
TATGTTCAGTAGCCAGCAGGTAAGGCTGGGCACTCTGGACAGACTGCCGGGGATAACCCG
GAGGAAGGCGGGGATGACGTCAAATCATCATGCCCCTTACGGCCTGGGCTACACACGTGC
TACAATGGCGTAAACAAAGGGAAGCGAGAGGGTGACCTGGAGCGAATCCCAAAAATAACG
TCCCAGTTCGGACTGTAGTCTGCAACCCGACTACACGAAGCTGGAATCGCTAGTAATCGC
GAATCAGCATGTCGCGGTGAATACGTTCCCGGGTCTTGTACACACCGCCCGTCACACCAT
GGGAGTTGGAAATGCCCGAAGTCAGTGACCTAACCGCAAGGGAGGAGCTGCCGAAGGTGG
AGCCGATGACTGGGGTGAAGTCGTAACAAGGTAGCCGTATCGGAAGGTGCGGCTG
>Coprococcus comes
SEQ ID NO. 6
AGAGTTTGATCCTGGCTCAGGATGAACGCTGGCGGCGTGCTTAACACATGCAAGTCGAAC
GAAGCACTTTAACCTGATTCTTCGGATGAAGGTTTTTGTGACTGAGTGGCGGACGGGTGA
GTAACGCGTGGGTAACCTGCCTCATACAGGGGGATAACAGTTAGAAATGACTGCTAATAC
CGCATAAGACCACGGAGCCGCATGGCTCAGTGGGAAAAACTCCGGTGGTATGAGATGGAC
CCGCGTCTGATTAGGTAGTTGGTGGGGTAACGGCCTACCAAGCCAACGATCAGTAGCCGA
CCTGAGAGGGTGACCGGCCACATTGGGACTGAGACACGGCCCAAACTCCTACGGGAGGCA
GCAGTGGGGAATATTGCACAATGGGGGAAACCCTGATGCAGCGACGCCGCGTGAGCGAAG
AAGTATTTCGGTATGTAAAGCTCTATCAGCAGGGAAGAAAATGACGGTACCTGACTAAGA
AGCACCGGCTAAATACGTGCCAGCAGCCGCGGTAATACGTATGGTGCAAGCGTTATCCGG
ATTTACTGGGTGTAAAGGGAGCGTAGACGGCTGTGTAAGTCTGAAGTGAAAGCCCGGGGC
TCAACCCCGGGACTGCTTTGGAAACTATGCAGCTAGAGTGTCGGAGAGGTAAGTGGAATT
CCCAGTGTAGCGGTGAAATGCGTAGATATTGGGAGGAACACCAGTGGCGAAGGCGGCTTA
CTGGACGATGACTGACGTTGAGGCTCGAAAGCGTGGGGAGCAAACAGGATTAGATACCCT
GGTAGTCCACGCCGTAAACGATGACTACTAGGTGTCGGGGAGCAAAGCTCTTCGGTGCCG
CAGCAAACGCAATAAGTAGTCCACCTGGGGAGTACGTTCGCAAGAATGAAACTCAAAGGA
ATTGACGGGGACCCGCACAAGCGGTGGAGCATGTGGTTTAATTCGAAGCAACGCGAAGAA
CCTTACCTGCTCTTGACATCCCGGTGACCGGCGTGTAATGACGCCTTTTCTTCGGAACAC
CGGTGACAGGTGGTGCATGGTTGTCGTCAGCTCGTGTCGTGAGATGTTGGGTTAAGTCCC
GCAACGAGCGCAACCCTTATCTTCAGTAGCCAGCAATTCGGATGGGCACTCTGGAGAGAC
TGCCAGGGATAACCTGGAGGAAGGTGGGGATGACGTCAAATCATCATGCCCCTTATGAGC
AGGGCTACACACGTGCTACAATGGCGTAAACAAAGGGAAGCGAGCCTGCGAGGGTAAGCA
AATCTCAAAAATAACGTCTCAGTTCGGATTGTAGTCTGCAACTCGACTACATGAAGCTGG
AATCGCTAGTAATCGCGAATCAGCATGTCGCGGTGAATACGTTCCCGGGTCTTGTACACA
CCGCCCGTCACACCATGGGAGTTGGTAACGCCCGAAGTCAGTGACCCAACCGTAAGGAGG
GAGCTGCCGAAGGTGGGACCGATAACTGGGGTGAAGTCGTAACAAGGTAGCCGTATCGGA
AGGTGCGGCTG
>Dorea sp.
SEQ ID NO. 7
AGAGTTTGATCCTGGCTCAGGATGAACGCTGGCGGCGTGCTTAACACATGCAAGTCGAGC
GAAGCACTTAAGTTTGATTCTTCGGATGAAGACTTTTGTGACTGAGCGGCGGACGGGTGA
GTAACGCGTGGGTAACCTGCCTCATACAGGGGGATAACAGTTAGAAATGGCTGCTAATAC
CGCATAAGACCACAGTACTGCATGGTACAGTGGTAAAAACTCCGGTGGTATGAGATGGAC
CCGCGTCTGATTAGGTAGTTGGTGAGGTAACGGCCCACCAAGCCGACGATCAGTAGCCGA
CCTGAGAGGGTGACCGGCCACATTGGGACTGAGACACGGCCCAGACTCCTACGGGAGGCA
GCAGTGGGGAATATTGCACAATGGGCGAAAGCCTGATGCAGCGACGCCGCGTGAAGGATG
AAGTATTTCGGTATGTAAACTTCTATCAGCAGGGAAGAAAATGACGGTACCTGACTAAGA
AGCCCCGGCTAACTACGTGCCAGCAGCCGCGGTAATACGTAGGGGGCAAGCGTTATCCGG
ATTTACTGGGTGTAAAGGGAGCGTAGACGGCTGTGCAAGTCTGAAGTGAAAGGCATGGGC
TCAACCTGTGGACTGCTTTGGAAACTGTGCAGCTAGAGTGTCGGAGAGGTAAGTGGAATT
CCTAGTGTAGCGGTGAAATGCGTAGATATTAGGAGGAACACCAGTGGCGAAGGCGGCTTA
CTGGACGATGACTGACGTTGAGGCTCGAAAGCGTGGGGAGCAAACAGGATTAGATACCCT
GGTAGTCCACGCCGTAAACGATGACTGCTAGGTGTCGGGTAGCAAAGCTATTCGGTGCCG
CAGCTAACGCAATAAGCAGTCCACCTGGGGAGTACGTTCGCAAGAATGAAACTCAAAGGA
ATTGACGGGGACCCGCACAAGCGGTGGAGCATGTGGTTTAATTCGAAGCAACGCGAAGAA
CCTTACCTGATCTTGACATCCCGATGACCGCTTCGTAATGGAAGTTTTTCTTCGGAACAT
CGGTGACAGGTGGTGCATGGTTGTCGTCAGCTCGTGTCGTGAGATGTTGGGTTAAGTCCC
GCAACGAGCGCAACCCTTATCTTCAGTAGCCAGCATTTAGGATGGGCACTCTGGAGAGAC
TGCCAGGGATAACCTGGAGGAAGGTGGGGATGACGTCAAATCATCATGCCCCTTATGACC
AGGGCTACACACGTGCTACAATGGCGTAAACAAAGGGAAGCAGAGCCGCGAGGCCGAGCA
AATCTCAAAAATAACGTCTCAGTTCGGATTGTAGTCTGCAACTCGACTACATGAAGCTGG
AATCGCTAGTAATCGCAGATCAGAATGCTGCGGTGAATACGTTCCCGGGTCTTGTACACA
CCGCCCGTCACACCATGGGAGTCAGTAACGCCCGAAGTCAGTGACCCAACCGAAAGGAGG
GAGCTGCCGAAGGTGGGACCGATAACTGGGGTGAAGTCGTAACAAGGTAGCCGTATCGGA
AGGTGCGGCTG
>Erysipelotrichaceae UCG-003 sp.
SEQ ID NO. 8
AGAGTTTGATCCTGGCTCAGGATGAACGCTGGCGGCGTGCCTAATACATGCAAGTCGAAC
GCTTCACTTCGGTGAAGAGTGGCGAACGGGTGAGTAATACATAAGTAACCTGGCATCTAC
AGGGGGATAACTGATGGAAACGTCAGCTAAGACCGCATAGGTGTAGAGATCGCATGAACT
CTATATGAAAAGTGCTACGGGACTGGTAGATGATGGACTTATGGCGCATTAGCTTGTTGG
TAGGGTAACGGCCTACCAAGGCGACGATGCGTAGCCGACCTGAGAGGGTGACCGGCCACA
CTGGGACTGAGACACGGCCCAGACTCCTACGGGAGGCAGCAGTAGGGAATTTTCGGCAAT
GGGGGAAACCCTGACCGAGCAACGCCGCGTGAAGGAAGAAGTAATTCGTTATGTAAACTT
CTGTCATAGAGGAAGAACGGTGGATATAGGGAATGATATCCAAGTGACGGTACTCTATAA
GAAAGCCACGGCTAACTACGTGCCAGCAGCCGCGGTAATACGTAGGTGGCGAGCGTTATC
CGGAATTATTGGGCGTAAAGAGGGAGCAGGCGGCACTAAGGGTCTGTGGTGAAAGATCGA
AGCTTAACTTCGGTAAGCCATGGAAACCGTAGAGCTAGAGTGTGTGAGAGGATCGTGGAA
TTCCATGTGTAGCGGTGAAATGCGTAGATATATGGAGGAACACCAGTGGCGAAGGCGACG
ATCTGGCGCATAACTGACGCTCAGTCCCGAAAGCGTGGGGAGCAAATAGGATTAGATACC
CTAGTAGTCCACGCCGTAAACGATGAGTACTAAGTGTTGGGGGTCAAACCTCAGTGCTGC
AGTTAACGCAATAAGTACTCCGCCTGAGTAGTACGTTCGCAAGAATGAAACTCAAAGGAA
TTGACGGGGGCCCGCACAAGCGGTGGAGCATGTGGTTTAATTCGAAGCAACGCGAAGAAC
CTTACCAGGTCTTGACATCGATCTAAAGGCTCCAGAGATGGAGAGATAGCTATAGAGAAG
ACAGGTGGTGCATGGTTGTCGTCAGCTCGTGTCGTGAGATGTTGGGTTAAGTCCCGCAAC
GAGCGCAACCCCTGTTGCCAGTTGCCAGCATTAAGTTGGGGACTCTGGCGAGACTGCCGG
TGACAAGCCGGAGGAAGGCGGGGATGACGTCAAATCATCATGCCCCTTATGACCTGGGCT
ACACACGTGCTACAATGGACAGAGCAGAGGGAAGCGAAGCCGCGAGGTGGAGCGAAACCC
ATAAAACTGTTCTCAGTTCGGACTGCAGTCTGCAACTCGACTGCACGAAGATGGAATCGC
TAGTAATCGCGAATCAGCATGTCGCGGTGAATACGTTCTCGGGCCTTGTACACACCGCCC
GTCACACCATGAGAGTCGGTAACACCCGAAGCCGGTGGCCTAACCGCAAGGAAGGAGCTG
TCTAAGGTGGGACTGATGATTGGGGTGAAGTCGTAACAAGGTATCCCTACGGGAACGTGG
GGATG
>Odoribacter splanchnicus
SEQ ID NO. 9
AGAGTTTGATCCTGGCTCAGGATGAACGCTAGCGACAGGCTTAACACATGCAAGTCGAGG
GGCATCATGAGGTAGCAATACCTTGATGGCGACCGGCGCACGGGTGAGTAACGCGTATGC
AACCTGCCCGATACCGGGGTATAGCCCATGGAAACGTGGATTAACACCCCATAGTACTTT
TATCCTGCCTGGGATGGGAGTTAAATGTTCAAGGTATCGGATGGGCATGCGTCCTATTAG
TTAGTTGGCGGGGTAACAGCCCACCAAGACGATGATAGGTAGGGGTTCTGAGAGGAAGGT
CCCCCACATTGGAACTGAGACACGGTCCAAACTCCTACGGGAGGCAGCAGTGAGGAATAT
TGGTCAATGGACGTAAGTCTGAACCAGCCAAGTCGCGTGAGGGAAGACTGCCCTATGGGT
TGTAAACCTCTTTTATAAGGGAAGAATAAGTTCTACGTGTAGAATGATGCCTGTACCTTA
TGAATAAGCATCGGCTAACTCCGTGCCAGCAGCCGCGGTAATACGGAGGATGCGAGCGTT
ATCCGGATTTATTGGGTTTAAAGGGTGCGTAGGCGGTTTATTAAGTTAGTGGTTAAATAT
TTGAGCTAAACTCAATTGTGCCATTAATACTGGTAAACTGGAGTACAGACGAGGTAGGCG
GAATAAGTTAAGTAGCGGTGAAATGCATAGATATAACTTAGAACTCCGATAGCGAAGGCA
GCTTACCAGACTGAAACTGACGCTGATGCACGAGAGCGTGGGTAGCGAACAGGATTAGAT
ACCCTGGTAGTCCACGCCGTAAACGATGCTCACTGGTTCTGTGCGATATATTGTACGGGA
TTAAGCGAAAGTATTAAGTGAGCCACCTGGGGAGTACGTCGGCAACGATGAAACTCAAAG
GAATTGACGGGGGCCCGCACAAGCGGAGGAACATGTGGTTTAATTCGATGATACGCGAGG
AACCTTACCTGGGTTTAAATGGGAAATGTCGTATTTGGAAACAGATATTCTCTTCGGAGC
GTTTTTCAAGGTGCTGCATGGTTGTCGTCAGCTCGTGCCGTGAGGTGTCGGGTTAAGTCC
CATAACGAGCGCAACCCTTACCGTTAGTTGCTAGCATGTAATGATGAGCACTCTAACGGG
ACTGCCACCGTAAGGTGAGAGGAAGGCGGGGATGACGTCAAATCAGCACGGCCCTTACAC
CCAGGGCTACACACGTGTTACAATGGCCGGTACAGAGGGCCGCTACCAGGTGACTGGATG
CCAATCTCAAAAGCCGGTCGTAGTTCGGATTGGAGTCTGTAACCCGACTCCATGAAGTTG
GATTCGCTAGTAATCGCGCATCAGCCATGGCGCGGTGAATACGTTCCCGGGCCTTGTACA
CACCGCCCGTCAAGCCATGGAAGCCGGGGGTGCCTGAAGTCCGTAACCGCGAGGATCGGC
CTAGGGCAAAACTGGTAACTGGGGCTAAGTCGTAACAAGGTAGCCGTACCGGAAGGTGCG
GCTG
>Parabacteroides distasonis
SEQ ID NO. 10
AGAGTTTGATCCTGGCTCAGGATGAACGCTAGCGACAGGCTTAACACATGCAAGTCGAGG
GGCAGCACAGGTAGCAATACCGGGTGGCGACCGGCGCACGGGTGAGTAACGCGTATGCAA
CTTACCTATCAGAGGGGGATAACCCGGCGAAAGTCGGACTAATACCGCATGAAGCAGGGG
CCCCGCATGGGGATATTTGCTAAAGATTCATCGCTGATAGATAGGCATGCGTTCCATTAG
GCAGTTGGCGGGGTAACGGCCCACCAAACCGACGATGGATAGGGGTTCTGAGAGGAAGGT
CCCCCACATTGGTACTGAGACACGGACCAAACTCCTACGGGAGGCAGCAGTGAGGAATAT
TGGTCAATGGGCGTAAGCCTGAACCAGCCAAGTCGCGTGAGGGATGAAGGTTCTATGGAT
CGTAAACCTCTTTTATAAGGGAATAAAGTGCGGGACGTGTCCTGTTTTGTATGTACCTTA
TGAATAAGGATCGGCTAACTCCGTGCCAGCAGCCGCGGTAATACGGAGGATCCGAGCGTT
ATCCGGATTTATTGGGTTTAAAGGGTGCGTAGGCGGCCTTTTAAGTCAGCGGTGAAAGTC
TGTGGCTCAACCATAGAATTGCCGTTGAAACTGGGGGGCTTGAGTATGTTTGAGGCAGGC
GGAATGCGTGGTGTAGCGGTGAAATGCTTAGATATCACGCAGAACCCCGATTGCGAAGGC
AGCCTGCCAAGCCATGACTGACGCTGATGCACGAAAGCGTGGGGATCAAACAGGATTAGA
TACCCTGGTAGTCCACGCAGTAAACGATGATCACTAGCTGTTTGCGATACAGTGTAAGCG
GCACAGCGAAAGCGTTAAGTGATCCACCTGGGGAGTACGCCGGCAACGGTGAAACTCAAA
GGAATTGACGGGGGCCCGCACAAGCGGAGGAACATGTGGTTTAATTCGATGATACGCGAG
GAACCTTACCCGGGTTTGAACGCATTCGGACCGAGGTGGAAACACCTTTTCTAGCAATAG
CCGTTTGCGAGGTGCTGCATGGTTGTCGTCAGCTCGTGCCGTGAGGTGTCGGCTTAAGTG
CCATAACGAGCGCAACCCTTGCCACTAGTTACTAACAGGTGATGCTGAGGACTCTGGTGG
GACTGCCAGCGTAAGCTGCGAGGAAGGCGGGGATGACGTCAAATCAGCACGGCCCTTACA
TCCGGGGCGACACACGTGTTACAATGGCGTGGACAAAGGGATGCCACCTGGCGACAGGGA
GCGAATCCCCAAACCACGTCTCAGTTCGGATCGGAGTCTGCAACCCGACTCCGTGAAGCT
GGATTCGCTAGTAATCGCGCATCAGCCATGGCGCGGTGAATACGTTCCCGGGCCTTGTAC
ACACCGCCCGTCAAGCCATGGGAGCCGGGGGTACCTGAAGTCCGTAACCGAAAGGATCGG
CCTAGGGTAAAACTGGTGACTGGGGCTAAGTCGTAACAAGGTAGCCGTACCGGAAGGTGC
GGCTG
>Parabacteroides distasonis
SEQ ID NO. 11
AGAGTTTGATCCTGGCTCAGGATGAACGCTAGCGACAGGCTTAACACATGCAAGTCGAGG
GGCAGCACAGGTAGCAATACCGGGTGGCGACCGGCGCACGGGTGAGTAACGCGTATGCAA
CTTACCTATCAGAGGGGGATAACCCGGCGAAAGTCGGACTAATACCGCATGAAGCAGGGG
CCCCGCATGGGGATATTTGCTAAAGATTCATCGCTGATAGATAGGCATGCGTTCCATTAG
GCAGTTGGCGGGGTAACGGCCCACCAAACCGACGATGGATAGGGGTTCTGAGAGGAAGGT
CCCCCACATTGGTACTGAGACACGGACCAAACTCCTACGGGAGGCAGCAGTGAGGAATAT
TGGTCAATGGGCGTAAGCCTGAACCAGCCAAGTCGCGTGAGGGATGAAGGTTCTATGGAT
CGTAAACCTCTTTTATAAGGGAATAAAGTGCGGGACGTGTCCCGTTTTGTATGTACCTTA
TGAATAAGGATCGGCTAACTCCGTGCCAGCAGCCGCGGTAATACGGAGGATCCGAGCGTT
ATCCGGATTTATTGGGTTTAAAGGGTGCGTAGGCGGCCTTTTAAGTCAGCGGTGAAAGTC
TGTGGCTCAACCATAGAATTGCCGTTGAAACTGGGGGGCTTGAGTATGTTTGAGGCAGGC
GGAATGCGTGGTGTAGCGGTGAAATGCTTAGATATCACGCAGAACCCCGATTGCGAAGGC
AGCCTGCCAAGCCATGACTGACGCTGATGCACGAAAGCGTGGGGATCAAACAGGATTAGA
TACCCTGGTAGTCCACGCAGTAAACGATGATCACTAGCTGTTTGCGATACAGTGTAAGCG
GCACAGCGAAAGCGTTAAGTGATCCACCTGGGGAGTACGCCGGCAACGGTGAAACTCAAA
GGAATTGACGGGGGCCCGCACAAGCGGAGGAACATGTGGTTTAATTCGATGATACGCGAG
GAACCTTACCCGGGTTTGAACGCATTCGGACCGAGGTGGAAACACCTTTTCTAGCAATAG
CCGTTTGCGAGGTGCTGCATGGTTGTCGTCAGCTCGTGCCGTGAGGTGTCGGCTTAAGTG
CCATAACGAGCGCAACCCTTGCCACTAGTTACTAACAGGTTAGGCTGAGGACTCTGGTGG
GACTGCCAGCGTAAGCTGCGAGGAAGGCGGGGATGACGTCAAATCAGCACGGCCCTTACA
TCCGGGGCGACACACGTGTTACAATGGCGTGGACAAAGGGAAGCCACCTGGCGACAGGGA
GCGAATCCCCAAACCACGTCTCAGTTCGGATCGGAGTCTGCAACCCGACTCCGTGAAGCT
GGATTCGCTAGTAATCGCGCATCAGCCATGGCGCGGTGAATACGTTCCCGGGCCTTGTAC
ACACCGCCCGTCAAGCCATGGGAGCCGGGGGTACCTGAAGTCCGTAACCGCGAGGATCGG
CCTAGGGTAAAACTGGTGACTGGGGCTAAGTCGTAACAAGGTAGCCGTACCGGAAGGTGC
GGCTG
>Ruminiclostridium 9 sp.
SEQ ID NO. 12
AGAGTTTGATCCTGGCTCAGGATGAACGCTGGCGGCGTGCTTAACACATGCAAGTCGAAC
GGAACTCCTATGATTGAGGTTTCGGCCAAAAGAATAGGATGTTTAGTGGCGGACGGGTGA
GTAACGCGTGAGTAACCTGCCTTGGAGTGGGGAATAACACAGTGAAAACTGTGCTAATAC
CGCATAATGCATTTGGGTCGCATGATTCTGAATGCCAAAGATTTATCGCTCTGAGATGGA
CTCGCGTCTGATTAGCTAGTTGGCGGGGTAACGGCCCACCAAGGCGACGATCAGTAGCCG
GACTGAGAGGTTGGCCGGCCACATTGGGACTGAGACACGGCCCAGACTCCTACGGGAGGC
AGCAGTGGGGAATATTGGGCAATGGGCGCAAGCCTGACCCAGCAACGCCGCGTGAAGGAA
GAAGGCTTTCGGGTTGTAAACTTCTTTTGTCAGGGACGAAGCAAGTGACGGTACCTGACG
AATAAGCCACGGCTAACTACGTGCCAGCAGCCGCGGTAATACGTAGGTGGCAAGCGTTAT
CCGGATTTACTGGGTGTAAAGGGCGTGTAGGCGGGAAAGCAAGTCAGATGTGAAAACTGT
GGGCTCAACCCACAGCCTGCATTTGAAACTGTTTTTCTTGAGTACTGGAGAGGCAGATGG
AATTCCTAGTGTAGCGGTGAAATGCGTAGATATTAGGAGGAACACCAGTGGCGAAGGCGA
TCTGCTGGACAGCAACTGACGCTGAGGCGCGAAAGCGTGGGGAGCAAACAGGATTAGATA
CCCTGGTAGTCCACGCTGTAAACGATGGATACTAGGTGTGGGGGGTCTGACCCCCTCCGT
GCCGCAGTTAACACAATAAGTATCCCACCTGGGGAGTACGATCGCAAGGTTGAAACTCAA
AGGAATTGACGGGGGCCCGCACAAGCGGTGGAGTATGTGGTTTAATTCGAAGCAACGCGA
AGAACCTTACCAGGGCTTGACATCCCGGTGACCGGCTTAGAGATAAGCCTTTCTCTTCGG
AGACACTGGAGACAGGTGGTGCATGGTTGTCGTCAGCTCGTGTCGTGAGATGTTGGGTTA
AGTCCCGCAACGAGCGCAACCCTTATTGTTAGTTGCTACGCAAGAGCACTCTAGCGAGAC
TGCCGTTGACAAAACGGAGGAAGGTGGGGACGACGTCAAATCATCATGCCCCTTATGTCC
TGGGCCACACACGTACTACAATGGTGGTCAACAGAGGGAAGCAAGACTGTGAAGTGGAGC
AAACCCCTAAAAGCCATCCCAGTTCGGATCGCAGGCTGCAACCCGCCTGCGTGAAGTTGG
AATCGCTAGTAATCGCGGATCAGCATGCCGCGGTGAATACGTTCCCGGGCCTTGTACACA
CCGCCCGTCACACCATGAGAGTCGGGAACACCCGAAGTCCGTAGCCTAACCGCAAGGAGG
GCGCGGCCGAAGGTGGGTTCGATAATTGGGGTGAAGTCGTAACAAGGTAGCCGTATCGGA
AGGTGCGGCTG
>Ruminococcus torques
SEQ ID NO. 13
AGAGTTTGATCCTGGCTCAGGATGAACGCTGGCGGCGTGCCTAACACATGCAAGTCGAGC
GAAGCACTTTGCTTAGATTCTTCGGATGAAGAGGATTGTGACTGAGCGGCGGACGGGTGA
GTAACGCGTGGGTAACCTGCCTCATACAGGGGGATAACAGTTAGAAATGACTGCTAATAC
CGCATAAGACCACAGCACCGCATGGTGCGGGGGTAAAAACTCCGGTGGTATGAGATGGAC
CCGCGTCTGATTAGCTAGTTGGTAAGGTAACGGCTTACCAAGGCGACGATCAGTAGCCGA
CCTGAGAGGGTGACCGGCCACATTGGGACTGAGACACGGCCCAAACTCCTACGGGAGGCA
GCAGTGGGGAATATTGCACAATGGGGGAAACCCTGATGCAGCGACGCCGCGTGAGCGAAG
AAGTATTTCGGTATGTAAAGCTCTATCAGCAGGGAAGAAAATGACGGTACCTGACTAAGA
AGCACCGGCTAAATACGTGCCAGCAGCCGCGGTAATACGTATGGTGCAAGCGTTATCCGG
ATTTACTGGGTGTAAAGGGAGCGTAGACGGATGGGCAAGTCTGATGTGAAAACCCGGGGC
TCAACCCCGGGACTGCATTGGAAACTGTTCATCTAGAGTGCTGGAGAGGTAAGTGGAATT
CCTAGTGTAGCGGTGAAATGCGTAGATATTAGGAGGAACACCAGTGGCGAAGGCGGCTTA
CTGGACAGTAACTGACGTTGAGGCTCGAAAGCGTGGGGAGCAAACAGGATTAGATACCCT
GGTAGTCCACGCCGTAAACGATGACTACTAGGTGTCGGGTGGCAAAGCCATTCGGTGCCG
CAGCAAACGCAATAAGTAGTCCACCTGGGGAGTACGTTCGCAAGAATGAAACTCAAAGGA
ATTGACGGGGACCCGCACAAGCGGTGGAGCATGTGGTTTAATTCGAAGCAACGCGAAGAA
CCTTACCTGCTCTTGACATCCCGCTGACCGGACGGTAATGCGTCCTTCCCTTCGGGGCAG
CGGAGACAGGTGGTGCATGGTTGTCGTCAGCTCGTGTCGTGAGATGTTGGGTTAAGTCCC
GCAACGAGCGCAACCCCTATCTTTAGTAGCCAGCGGCCAGGCCGGGCACTCTAGAGAGAC
TGCCGGGGATAACCCGGAGGAAGGTGGGGATGACGTCAAATCATCATGCCCCTTATGAGC
AGGGCTACACACGTGCTACAATGGCGTAAACAAAGGGAAGCGAGACCGCGAGGTGGAGCA
AATCCCAAAAATAACGTCTCAGTTCGGATTGTAGTCTGCAACTCGACTACATGAAGCTGG
AATCGCTAGTAATCGCGAATCAGAATGTCGCGGTGAATACGTTCCCGGGTCTTGTACACA
CCGCCCGTCACACCATGGGAGTCAGTAACGCCCGAAGTCAGTGACCCAACCGTAAGGAGG
GAGCTGCCGAAGGCGGGACCGATAACTGGGGTGAAGTCGTAACAAGGTAGCCGTATCGGA
AGGTGCGGCTG
>Bacteroides fragilis
SEQ ID NO. 14
AGAGTTTGATCCTGGCTCAGGATGAACGCTAGCTACAGGCTTAACACATGCAAGTCGAGG
GGCATCAGGAAGAAAGCTTGCTTTCTTTGCTGGCGACCGGCGCACGGGTGAGTAACACGT
ATCCAACCTGCCCTTTACTCGGGGATAGCCTTTCGAAAGAAAGATTAATACCCGATAGCA
TAATGATTCCGCATGGTTTCATTATTAAAGGATTCCGGTAAAGGATGGGGATGCGTTCCA
TTAGGTTGTTGGTGAGGTAACGGCTCACCAAGCCTTCGATGGATAGGGGTTCTGAGAGGA
AGGTCCCCCACATTGGAACTGAGACACGGTCCAAACTCCTACGGGAGGCAGCAGTGAGGA
ATATTGGTCAATGGGCGCTAGCCTGAACCAGCCAAGTAGCGTGAAGGATGAAGGCTCTAT
GGGTCGTAAACTTCTTTTATATAAGAATAAAGTGCAGTATGTATACTGTTTTGTATGTAT
TATATGAATAAGGATCGGCTAACTCCGTGCCAGCAGCCGCGGTAATACGGAGGATCCGAG
CGTTATCCGGATTTATTGGGTTTAAAGGGAGCGTAGGTGGACTGGTAAGTCAGTTGTGAA
AGTTTGCGGCTCAACCGTAAAATTGCAGTTGATACTGTCAGTCTTGAGTACAGTAGAGGT
GGGCGGAATTCGTGGTGTAGCGGTGAAATGCTTAGATATCACGAAGAACTCCGATTGCGA
AGGCAGCTCACTGGACTGCAACTGACACTGATGCTCGAAAGTGTGGGTATCAAACAGGAT
TAGATACCCTGGTAGTCCACACAGTAAACGATGAATACTCGCTGTTTGCGATATACAGTA
AGCGGCCAAGCGAAAGCATTAAGTATTCCACCTGGGGAGTACGCCGGCAACGGTGAAACT
CAAAGGAATTGACGGGGGCCCGCACAAGCGGAGGAACATGTGGTTTAATTCGATGATACG
CGAGGAACCTTACCCGGGCTTAAATTGCAGTGGAATGATGTGGAAACATGTCAGTGAGCA
ATCACCGCTGTGAAGGTGCTGCATGGTTGTCGTCAGCTCGTGCCGTGAGGTGTCGGCTTA
AGTGCCATAACGAGCGCAACCCTTATCTTCAGTTACTAACAGGTCATGCTGAGGACTCTG
GAGAGACTGCCGTCGTAAGATGTGAGGAAGGTGGGGATGACGTCAAATCAGCACGGCCCT
TACGTCCGGGGCTACACACGTGTTACAATGGGGGGTACAGAAGGCAGCTAGCGGGTGACC
GTATGCTAATCCCAAAAGCCTCTCTCAGTTCGGATCGAAGTCTGCAACCCGACTTCGTGA
AGCTGGATTCGCTAGTAATCGCGCATCAGCCACGGCGCGGTGAATACGTTCCCGGGCCTT
GTACACACCGCCCGTCAAGCCATGGGAGCCGGGGGTACCTGAAGTACGTAACCGCAAGGA
TCGTCCTAGGGTAAAACTGGTGACTGGGGCTAAGTCGTAACAAGGTAGCCGTACCGGAAG
GTGCGGCTG
>Bacteroides fragilis
SEQ ID NO. 15
AGAGTTTGATCCTGGCTCAGGATGAACGCTAGCTACAGGCTTAACACATGCAAGTCGAGG
GGCATCAGGAAGAAAGCTTGCTTTCTTTGCTGGCGACCGGCGCACGGGTGAGTAACACGT
ATCCAACCTGCCCTTTACTCGGGGATAGCCTTTCGAAAGAAAGATTAATACCCGATGGCA
TAATGATTCCGCATGGTTTCATTATTAAAGGATTCCGGTAAAGGATGGGGATGCGTTCCA
TTAGGTTGTTGGTGAGGTAACGGCTCACCAAGCCTTCGATGGATAGGGGTTCTGAGAGGA
AGGTCCCCCACATTGGAACTGAGACACGGTCCAAACTCCTACGGGAGGCAGCAGTGAGGA
ATATTGGTCAATGGGCGTTAGCCTGAACCAGCCAAGTAGCGTGAAGGATGAAGGCTCTAT
GGGTCGTAAACTTCTTTTATATAAGAATAAAGTGCAGTATGTATACTGTTTTGTATGTAT
TATATGAATAAGGATCGGCTAACTCCGTGCCAGCAGCCGCGGTAATACGGAGGATCCGAG
CGTTATCCGGATTTATTGGGTTTAAAGGGAGCGTAGGTGGACTGGTAAGTCAGTTGTGAA
AGTTTGCGGCTCAACCGTAAAATTGCAGTTGATACTGTCAGTCTTGAGTACAGTAGAGGT
GGGCGGAATTCGTGGTGTAGCGGTGAAATGCTTAGATATCACGAAGAACTCCGATTGCGA
AGGCAGCTCACTGGACTGCAACTGACACTGATGCTCGAAAGTGTGGGTATCAAACAGGAT
TAGATACCCTGGTAGTCCACACAGTAAACGATGAATACTCGCTGTTTGCGATATACAGTA
AGCGGCCAAGCGAAAGCATTAAGTATTCCACCTGGGGAGTACGCCGGCAACGGTGAAACT
CAAAGGAATTGACGGGGGCCCGCACAAGCGGAGGAACATGTGGTTTAATTCGATGATACG
CGAGGAACCTTACCCGGGCTTAAATTGCAGTGGAATGATGTGGAAACATGTCAGTGAGCA
ATCACCGCTGTGAAGGTGCTGCATGGTTGTCGTCAGCTCGTGCCGTGAGGTGTCGGCTTA
AGTGCCATAACGAGCGCAACCCTTATCTTCAGTTACTAACAGGTTATGCTGAGGACTCTG
GAGAGACTGCCGTCGTAAGATGTGAGGAAGGTGGGGATGACGTCAAATCAGCACGGCCCT
TACGTCCGGGGCTACACACGTGTTACAATGGGGGGTACAGAAGGCAGCTAGCGGGTGACC
GTATGCTAATCCCAAAATCCTCTCTCAGTTCGGATCGAAGTCTGCAACCCGACTTCGTGA
AGCTGGATTCGCTAGTAATCGCGCATCAGCCACGGCGCGGTGAATACGTTCCCGGGCCTT
GTACACACCGCCCGTCAAGCCATGGGAGCCGGGGGTACCTGAAGTACGTAACCGCAAGGA
TCGTCCTAGGGTAAAACTGGTGACTGGGGCTAAGTCGTAACAAGGTAGCCGTACCGGAAG
GTGCGGCTG
>Blautia sp.
SEQ ID NO. 16
AGAGTTTGATCCTGGCTCAGGATGAACGCTGGCGGCGTGCTTAACACATGCAAGTCGAAC
GGGAAATACTTTATTGAAACTTCGGTCGATTTAATCTATTTCTAGTGGCGGACGGGTGAG
TAACGCGTGGGTAACCTGCCTTATACTGGGGGATAACAGCCAGAAATGACTGCTAATACC
GCATAAGCGCACGGGGCCGCATGGTCCTGTGTGAAAAACTCCGGTGGTATAAGATGGACC
CGCGTTGGATTAGCTAGTTGGCAGGGCAGCGGCCTACCAAGGCGACGATCCATAGCCGGC
CTGAGAGGGTGAACGGCCACATTGGGACTGAGACACGGCCCAGACTCCTACGGGAGGCAG
CAGTGGGGAATATTGCACAATGGGGGAAACCCTGATGCAGCGACGCCGCGTGAAGGAAGA
AGTATCTCGGTATGTAAACTTCTATCAGCAGGGAAGATAATGACGGTACCTGACTAAGAA
GCCCCGGCTAACTACGTGCCAGCAGCCGCGGTAATACGTAGGGGGCGAGCGTTATCCGGA
TTTACTGGGTGTAAAGGGAGCGTAGACGGCGTATCAAGTCTGATGTGAAAGGCAGGGGCT
TAACCCCTGGACTGCATTGGAAACTGGTATGCTTGAGTGCCGGAGGGGTAAGCGGAATTC
CTAGTGTAGCGGTGAAATGCGTAGATATTAGGAGGAACACCAGTGGCGAAGGCGGCTTAC
TGGACGGTAACTGACGTTGAGGCTCGAAAGCGTGGGGAGCAAACAGGATTAGATACCCTG
GTAGTCCACGCCGTAAACGATGAATACTAGGTGTCTGGGAGCACAGCTCTTAGGTGCCGC
CGCAAACGCATTAAGTATTCCACCTGGGGAGTACGTTCGCAAGAATGAAACTCAAAGGAA
TTGACGGGGACCCGCACAAGCGGTGGAGCATGTGGTTTAATTCGAAGCAACGCGAAGAAC
CTTACCAAATCTTGACATCCCTCTGACAGAGTATGTAATGTACTTTTCCTTCGGGACAGG
GGAGACAGGTGGTGCATGGTTGTCGTCAGCTCGTGTCGTGAGATGTTGGGTTAAGTCCCG
CAACGAGCGCAACCCCTATCCTTAGTAGCCAGCAAGTAATGTTGGGCACTCTGAGGAGAC
TGCCAGGGATAACCTGGAGGAAGGCGGGGATGACGTCAAATCATCATGCCCCTTATGATT
TGGGCTACACACGTGCTACAATGGCGTAAACAAAGGGAAGCGAACCTGTGAGGGTGGGCA
AATCTCAAAAATAACGTCCCAGTTCGGACTGCAGTCTGCAACTCGACTGCACGAAGCTGG
AATCGCTAGTAATCGCGGATCAGAATGCCGCGGTGAATACGTTCCCGGGTCTTGTACACA
CCGCCCGTCACACCATGGGAGTCAGTAACGCCCGAAGTCAGTGACCTAACCGTAAGGAAG
GAGCTGCCGAAGGCGGGACGGATGACTGGGGTGAAGTCGTAACAAGGTAGCCGTATCGGA
AGGTGCGGCTG
>Blautia sp.
SEQ ID NO. 17
AGAGTTTGATCCTGGCTCAGGATGAACGCTGGCGGCGTGCTTAACACATGCAAGTCGAAC
GGGGATTATTTCATTGAAGCTTCGGCAGATTTGGTTTAATCCTAGTGGCGGACGGGTGAG
TAACGCGTGGGTAACCTGCCTTATACAGGGGGATAACAGTCAGAAATGGCTGCTAATACC
GCATAAGCGCACAGGGCCGCATGGCCCGGTGTGAAAAACTGAGGTGGTATAAGATGGACC
CGCGTTGGATTAGCCAGTTGGCAGGGTAACGGCCTACCAAAGCGACGATCCATAGCCGGC
CTGAGAGGGTGAACGGCCACATTGGGACTGAGACACGGCCCAGACTCCTACGGGAGGCAG
CAGTGGGGAATATTGCACAATGGGGGAAACCCTGATGCAGCGACGCCGCGTGAAGGAAGA
AGTATCTCGGTATGTAAACTTCTATCAGCAGGGAAGAAAATGACGGTACCTGACTAAGAA
GCCCCGGCTAACTACGTGCCAGCAGCCGCGGTAATACGTAGGGGGCAAGCGTTATCCGGA
TTTACTGGGTGTAAAGGGAGCGTAGACGGCATAACAAGTCTGATGTGAAAGGCTGGGGCT
TAACCCCGGGACTGCATTGGAAACTGTTAAGCTTGAGTGCCGGAGGGGTAAGCGGAATTC
CTAGTGTAGCGGTGAAATGCGTAGATATTAGGAGGAACACCAGTGGCGAAGGCGGCTTAC
TGGACGGTAACTGACGTTGAGGCTCGAAAGCGTGGGGAGCAAACAGGATTAGATACCCTG
GTAGTCCACGCCGTAAACGATGAATACTAGGTGTCGGGGAGCACAGCTCTTCGGTGCCGC
CGCAAACGCATTAAGTATTCCACCTGGGGAGTACGTTCGCAAGAATGAAACTCAAAGGAA
TTGACGGGGACCCGCACAAGCGGTGGAGCATGTGGTTTAATTCGAAGCAACGCGAAGAAC
CTTACCAAGTCTTGACATCTGCCTGACCGGTGAGTAACGTCACCTTTCCTTCGGGACAGG
CAAGACAGGTGGTGCATGGTTGTCGTCAGCTCGTGTCGTGAGATGTTGGGTTAAGTCCCG
CAACGAGCGCAACCCCTATCCCCAGTAGCCAGCATGTAAAGGTGGGCACTCTGAGGAGAC
TGCCAGGGATAACCTGGAGGAAGGTGGGGATGACGTCAAATCATCATGCCCCTTATGATT
TGGGCTACACACGTGCTACAATGGCGTAAACAGAGGGAAGCGAAAGGGTGACCTGGAGCA
AATCCCAAAAATAACGTCCCAGTTCGGACTGTAGTCTGCAACCCGACTACACGAAGCTGG
AATCGCTAGTAATCGCGGATCAGAATGCCGCGGTGAATACGTTCCCGGGTCTTGTACACA
CCGCCCGTCACACCATGGGAGTCAGTAACGCCCGAAGTCAGTGACCTAACCGAAAGGGAG
GAGCTGCCGAAGGCGGGACGGATGACTGGGGTGAAGTCGTAACAAGGTAGCCGTATCGGA
AGGTGCGGCTG
>Lachnospiraceae FCS020
SEQ ID NO. 18
AGAGTTTGATCCTGGCTCAGGATGAACGCTGGCGGCGTGCTTAACACATGCAAGTCGAGC
GAAGCACTTATGGAGATTCTTCGGATGATCCATTTGTGACTGAGCGGCGGACGGGTGAGT
AACGCGTGAGTAACCTGCCTCATACAGGGGAATAACAGTTAGAAATGACTGCTAATGCCG
CATAAGCGCACAGGACCACATGGTCTGGTGTGAAAAACTCCGGTGGTATGAGATGGACTC
GCGTCTGATTAGCTAGTTGGTGAGGTAACGGCCCACCAAGGCGACGATCAGTAGCCGGCC
TGAGAGGGTGAACGGCCACATTGGGACTGAGACACGGCCCAAACTCCTACGGGAGGCAGC
AGTGGGGAATATTGCACAATGGGGGAAACCCTGATGCAGCGACGCCGCGTGAGTGAAGAA
GTATTTCGGTATGTAAAGCTCTATCAGCAGGGAAGAAAATGACGGTACCTGACTAAGAAG
CCCCGGCTAACTACGTGCCAGCAGCCGCGGTAATACGTAGGGGGCAAGCGTTATCCGGAT
TTACTGGGTGTAAAGGGAGCGTAGACGGCTTTGCAAGTCTGACGTGAAAATCCGGGGCTC
AACCCCGGAACTGCGTTGGAAACTGTGAGGCTTGAGTGCCGGAGAGGTAAGCGGAATTCC
TAGTGTAGCGGTGAAATGCGTAGATATTAGGAGGAACACCAGTGGCGAAGGCGGCTTACT
GGACGGCAACTGACGTTGAGGCTCGAAAGCGTGGGGAGCAAACAGGATTAGATACCCTGG
TAGTCCACGCGGTAAACGATGAATACTAGGTGTTGGGGGACAAAGTCCTTCGGTGCCGCC
GCAAACGCATTAAGTATTCCACCTGGGGAGTACGTTCGCAAGAATGAAACTCAAAGGAAT
TGACGGGGACCCGCACAAGCGGTGGAGCATGTGGTTTAATTCGAAGCAACGCGAAGAACC
TTACCAAGTCTTGACATCGATTCGCCGGGACTGTAATGAGTCCTTTCCCTTCGGGGACGA
AGAAGACAGGTGGTGCATGGTTGTCGTCAGCTCGTGTCGTGAGATGTTGGGTTAAGTCCC
GCAACGAGCGCAACCCTTATCTTCAGTAGCCAGCAAGTGAAGTTGGGCACTCTGGAGAGA
CTGCCAGGGACAACCTGGAGGAAGGTGGGGATGACGTCAAATCATCATGCCCCTTATGAC
TTGGGCTACACACGTGCTACAATGGCGTAAACAAAGGGAAGCAGCCCTGTGAAGGTGAGC
AAATCCCAAAAATAACGTCTCAGTTCGGATTGTAGTCTGCAACTCGACTACATGAAGCTG
GAATCGCTAGTAATCGCGAATCAGAATGTCGCGGTGAATACGTTCCCGGGTCTTGTACAC
ACCGCCCGTCACACCATGGGAGTCGGTAACGCCCGAAGTCAGTGACCCAACCGAAAGGAG
GGAGCTGCCGAAGGCGGGACTGGTAACTGGGGTGAAGTCGTAACAAGGTAGCCGTATCGG
AAGGTGCGGCTG
>Lachnoclostridium sp.
SEQ ID NO. 19
AGAGTTTGATCCTGGCTCAGGATGAACGCTGGCGGCGTGCCTAACACATGCAAGTCGAAC
GAAGCAATCAGAATGAAGTTTTCGGATGGATTTCTGATTGACTGAGTGGCGGACGGGTGA
GTAACGCGTGGATAACCTGCCTCACACTGGGGGATAACAGTTAGAAATGGCTGCTAATAC
CGCATAAGCGCACAGTACCGCATGGTACGGTGTGAAAAACTCCGGTGGTGTGAGATGGAT
CCGCGTCTGATTAGTTAGTTGGCGGGGTAACGGCCCACCAAGACAGCGATCAGTAGCCGA
CCTGAGAGGGTGACCGGCCACATTGGGACTGAGACACGGCCCAAACTCCTACGGGAGGCA
GCAGTGGGGAATATTGCACAATGGGCGAAAGCCTGATGCAGCGACGCCGCGTGAGTGAAG
AAGTATTTCGGTATGTAAAGCTCTATCAGCAGGGAAGAAACTGACGGTACCTGACTAAGA
AGCCCCGGCTAACTACGTGCCAGCAGCCGCGGTAATACGTAGGGGGCAAGCGTTATCCGG
ATTTACTGGGTGTAAAGGGAGCGTAGACGGCGAAGCAAGTCTGGAGTGAAAACCCAGGGC
TCAACCCTGGGACTGCTTTGGAAACTGTTTTGCTAGAGTGTCGGAGAGGTAAGTGGAATT
CCTAGTGTAGCGGTGAAATGCGTAGATATTAGGAGGAACACCAGTGGCGAAGGCGGCTTA
CTGGACGATAACTGACGTTGAGGCTCGAAAGCGTGGGGAGCAAACAGGATTAGATACCCT
GGTAGTCCACGCCGTAAACGATGAATGCTAGGTGTTGGGGGGCAAAGCCCTTCGGTGCCG
TCGCAAACGCAATAAGCATTCCACCTGGGGAGTACGTTCGCAAGAATGAAACTCAAAGGA
ATTGACGGGGACCCGCACAAGCGGTGGAGCATGTGGTTTAATTCGAAGCAACGCGAAGAA
CCTTACCAAGTCTTGACATCCCCCTGACCGGTCAGTAAAGTGACCTTTCCTTCGGGACAG
GGGAGACAGGTGGTGCATGGTTGTCGTCAGCTCGTGTCGTGAGATGTTGGGTTAAGTCCC
GCAACGAGCGCAACCCTTATCCTTAGTAGCCAGCAGGTAAAGCTGGGCACTCTAGGGAGA
CTGCCAGGGATAACCTGGAGGAAGGTGGGGATGACGTCAAATCATCATGCCCCTTATGAT
TTGGGCTACACACGTGCTACAATGGCGTAAACAAAGGGAAGCGACCCTGCGAAGGCAAGC
AAATCCCAAAAATAACGTCCCAGTTCGGACTGTAGTCTGCAACCCGACTACACGAAGCTG
GAATCGCTAGTAATCGCGAATCAGAATGTCGCGGTGAATACGTTCCCGGGTCTTGTACAC
ACCGCCCGTCACACCATGGGAGTCAGCAACGCCCGAAGTCAGTGACCCAACCGAAAGGAG
GGAGCTGCCGAAGGCGGGGCAGGTAACTGGGGTGAAGTCGTAACAAGGTAGCCGTATCGG
AAGGTGCGGCTG
>Bifidobacterium longum
SEQ ID NO. 20
AGGGTTCGATTCTGGCTCAGGATGAACGCTGGCGGCGTGCTTAACACATGCAAGTCGAAC
GGGATCCATCAGGCTTTGCTTGGTGGTGAGAGTGGCGAACGGGTGAGTAATGCGTGACCG
ACCTGCCCCATACACCGGAATAGCTCCTGGAAACGGGTGGTAATGCCGGATGCTCCAGTT
GATCGCATGGTCTTCTGGGAAAGCTTTCGCGGTATGGGATGGGGTCGCGTCCTATCAGCT
TGACGGCGGGGTAACGGCCCACCGTGGCTTCGACGGGTAGCCGGCCTGAGAGGGCGACCG
GCCACATTGGGACTGAGATACGGCCCAGACTCCTACGGGAGGCAGCAGTGGGGAATATTG
CACAATGGGCGCAAGCCTGATGCAGCGACGCCGCGTGAGGGATGGAGGCCTTCGGGTTGT
AAACCTCTTTTATCGGGGAGCAAGCGAGAGTGAGTTTACCCGTTGAATAAGCACCGGCTA
ACTACGTGCCAGCAGCCGCGGTAATACGTAGGGTGCAAGCGTTATCCGGAATTATTGGGC
GTAAAGGGCTCGTAGGCGGTTCGTCGCGTCCGGTGTGAAAGTCCATCGCTTAACGGTGGA
TCCGCGCCGGGTACGGGCGGGCTTGAGTGCGGTAGGGGAGACTGGAATTCCCGGTGTAAC
GGTGGAATGTGTAGATATCGGGAAGAACACCAATGGCGAAGGCAGGTCTCTGGGCCGTTA
CTGACGCTGAGGAGCGAAAGCGTGGGGAGCGAACAGGATTAGATACCCTGGTAGTCCACG
CCGTAAACGGTGGATGCTGGATGTGGGGCCCGTTCCACGGGTTCCGTGTCGGAGCTAACG
CGTTAAGCATCCCGCCTGGGGAGTACGGCCGCAAGGCTAAAACTCAAAGAAATTGACGGG
GGCCCGCACAAGCGGCGGAGCATGCGGATTAATTCGATGCAACGCGAAGAACCTTACCTG
GGCTTGACATGTTCCCGACGGTCGTAGAGATACGGCTTCCCTTCGGGGCGGGTTCACAGG
TGGTGCATGGTCGTCGTCAGCTCGTGTCGTGAGATGTTGGGTTAAGTCCCGCAACGAGCG
CAACCCTCGCCCCGTGTTGCCAGCGGATTATGCCGGGAACTCACGGGGGACCGCCGGGGT
TAACTCGGAGGAAGGTGGGGATGACGTCAGATCATCATGCCCCTTACGTCCAGGGCTTCA
CGCATGCTACAATGGCCGGTACAACGGGATGCGACGCGGCGACGCGGAGCGGATCCCTGA
AAACCGGTCTCAGTTCGGATCGCAGTCTGCAACTCGACTGCGTGAAGGCGGAGTCGCTAG
TAATCGCGAATCAGCAACGTCGCGGTGAATGCGTTCCCGGGCCTTGTACACACCGCCCGT
CAAGTCATGAAAGTGGGCAGCACCCGAAGCCGGTGGCCTAACCCCTTGTGGGATGGAGCC
GTCTAAGGTGAGGCTCGTGATTGGGACTAAGTCGTAACAAGGTAGCCGTACCGGAAGGTG
CGGCTG
>Bacteroides sp.
SEQ ID NO. 21
AGAGTTTGATCCTGGCTCAGGATGAACGCTAGCTACAGGCTTAACACATGCAAGTCGAGG
GGCAGCATTTTAGTTTGCTTGCAAACTAAAGATGGCGACCGGCGCACGGGTGAGTAACAC
GTATCCAACCTGCCGATAACTCGGGGATAGCCTTTCGAAAGAAAGATTAATATCCGATAG
TATATTAAAACCGCATGGTTTTACTATTAAAGAATTTCGGTTATCGATGGGGATGCGTTC
CATTAGTTTGTTGGCGGGGTAACGGCCCACCAAGACTACGATGGATAGGGGTTCTGAGAG
GAAGGTCCCCCACATTGGAACTGAGACACGGTCCAAACTCCTACGGGAGGCAGCAGTGAG
GAATATTGGTCAATGGACGAGAGTCTGAACCAGCCAAGTAGCGTGAAGGATGACTGCCCT
ATGGGTTGTAAACTTCTTTTATATGGGAATAAAGTATTCCACGTGTGGAATTTTGTATGT
ACCATATGAATAAGGATCGGCTAACTCCGTGCCAGCAGCCGCGGTAATACGGAGGATCCG
AGCGTTATCCGGATTTATTGGGTTTAAAGGGAGCGTAGGTGGATTGTTAAGTCAGTTGTG
AAAGTTTGCGGCTCAACCGTAAAATTGCAGTTGAAACTGGCAGTCTTGAGTACAGTAGAG
GTGGGCGGAATTCGTGGTGTAGCGGTGAAATGCTTAGATATCACGAAGAACTCCGATTGC
GAAGGCAGCTCACTAGACTGCAACTGACACTGATGCTCGAAAGTGTGGGTATCAAACAGG
ATTAGATACCCTGGTAGTCCACACAGTAAACGATGAATACTCGCTGTTTGCGATATACAG
TAAGCGGCCAAGCGAAAGCATTAAGTATTCCACCTGGGGAGTACGCCGGCAACGGTGAAA
CTCAAAGGAATTGACGGGGGCCCGCACAAGCGGAGGAACATGTGGTTTAATTCGATGATA
CGCGAGGAACCTTACCCGGGCTTAAATTGCATTTGAATAATCTGGAAACAGGTTAGCCGC
AAGGCAAATGTGAAGGTGCTGCATGGTTGTCGTCAGCTCGTGCCGTGAGGTGTCGGCTTA
AGTGCCATAACGAGCGCAACCCTTATCTTTAGTTACTAACAGGTCATGCTGAGGACTCTA
GAGAGACTGCCGTCGTAAGATGTGAGGAAGGTGGGGATGACGTCAAATCAGCACGGCCCT
TACGTCCGGGGCTACACACGTGTTACAATGGGGGGTACAGAAGGCAGCTACCTGGTGACA
GGATGCTAATCCCAAAAACCTCTCTCAGTTCGGATCGAAGTCTGCAACCCGACTTCGTGA
AGCTGGATTCGCTAGTAATCGCGCATCAGCCATGGCGCGGTGAATACGTTCCCGGGCCTT
GTACACACCGCCCGTCAAGCCATGAAAGCCGGGGGTACCTGAAGTACGTAACCGCAAGGA
GCGTCCTAGGGTAAAACTGGTAATTGGGGCTAAGTCGTAACAAGGTAGCCGTACCGGAAG
GTGCGGCTG

1. A composition comprising two or more isolated bacteria wherein said bacteria are selected from a bacterium having a 16sDNA of SEQ ID No. 1, 2 or 3 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 4 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 5 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 6 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 7 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 8 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 9 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of the following SEQ ID Nos. 10 or 11 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 12 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 13 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 14 or 15 or a 16S rDNA sequence having at least 90% e.g. at least 91%, 92%, 93%, 94%, 95%, 96%, 97%, 98%, 99%; e.g. 97% or 98.7% identity thereto, a bacterium having a 16sDNA of SEQ ID No. 16 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 17 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 18 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 19 or a 16S rDNA sequence having at least 90% e.g. at least 91%, 92%, 93%, 94%, 95%, 96%, 97%, 98%, 99%; e.g. 97% or 98.7% identity thereto; a bacterium having a 16sDNA of SEQ ID No. 20 or a 16S rDNA sequence having at least 90% e.g. at least 91%, 92%, 93%, 94%, 95%, 96%, 97%, 98%, 99%; e.g. 97% or 98.7% identity thereto and a bacterium having a 16sDNA of SEQ ID No. 21 or a 16S rDNA sequence having at least 90% e.g. at least 91%, 92%, 93%, 94%, 95%, 96%, 97%, 98%, 99%; e.g. 97% or 98.7% identity thereto.

2. The composition according to claim 1 comprising 2, 3, 4, 5, 6, 7, 8, 9, 10, 11, 12, 13, 14, 15, 16 or 17 isolated bacteria.

3. The composition according to claim 1 or 2 wherein the two or more isolated bacteria comprise 16S rDNA sequences having at least 97% sequence identity with a nucleic acid sequence selected from SEQ ID NOs: 1 to 21.

4. The composition according to a preceding claim comprising 15 isolated bacteria having a 16sDNA of the following SEQ ID Nos.:
SEQ ID No. 1 (or 2 or 3) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 6 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 7 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 8 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 9 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 10 (or 11) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 12 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 13 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 14 (or 15) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 16 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 17 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 18 or a 16S rDNA sequence having at least 90% e.g. at least 91%, 92%, 93%, 94%, 95%, 96%, 97%, 98%, 99%; e.g. 97% or 98.7% identity thereto and
SEQ ID No. 19 or a 16S rDNA sequence having at least 90% e.g. at least 91%, 92%, 93%, 94%, 95%, 96%, 97%, 98%, 99%; e.g. 97% or 98.7% identity thereto.

5. The composition according to any of claims 1 to 3 comprising 10 isolated bacteria having a 16sDNA of the following SEQ ID Nos.:
SEQ ID No. 1 (or 2 or 3) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 6 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 7 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 8 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 9 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 10 (or 11) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 12 or a 16S rDNA sequence having at least 90% e.g. at least 91%, 92%, 93%, 94%, 95%, 96%, 97%, 98%, 99%; e.g. 97% or 98.7% identity thereto and
SEQ ID No. 13 or a 16S rDNA sequence having at least 90% e.g. at least 91%, 92%, 93%, 94%, 95%, 96%, 97%, 98%, 99%; e.g. 97% or 98.7% identity thereto.

6. The composition according to any of claims 1 to 3 comprising 7 isolated bacteria having a 16sDNA of the following SEQ ID Nos.:
SEQ ID No. 1 (or 2 or 3) or a 16S rDNA sequence having at least 90% e.g. at least 91%, 92%, 93%, 94%, 95%, 96%, 97%, 98%, 99%; e.g. 97% or 98.7% identity thereto
SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 6 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 7 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 8 or a 16S rDNA sequence having at least 90% e.g. at least 91%, 92%, 93%, 94%, 95%, 96%, 97%, 98%, 99%; e.g. 97% or 98.7% identity thereto and
SEQ ID No. 9 or a 16S rDNA sequence having at least 90% e.g. at least 91%, 92%, 93%, 94%, 95%, 96%, 97%, 98%, 99%; e.g. 97% or 98.7% identity thereto.

7. The composition according to any of claims 1 to 3 comprising 5 isolated bacteria having a 16sDNA of the following SEQ ID Nos.:
SEQ ID No. 1 (or 2 or 3) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 4 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 5 or a 16S rDNA sequence having at least 90% e.g. at least 91%, 92%, 93%, 94%, 95%, 96%, 97%, 98%, 99%; e.g. 97% or 98.7% identity thereto,
SEQ ID No. 6 or a 16S rDNA sequence having at least 90% e.g. at least 91%, 92%, 93%, 94%, 95%, 96%, 97%, 98%, 99%; e.g. 97% or 98.7% identity thereto and
SEQ ID No. 7 or a 16S rDNA sequence having at least 90% e.g. at least 91%, 92%_{, 93}%_{, 94}%, 95%, 96%, 97%, 98%, 99%; e.g. 97% or 98.7% identity thereto.

8. The composition according to any of claims 1 to 3 comprising 4 isolated bacteria having a 16sDNA of the following SEQ ID Nos.:
SEQ ID No. 3 (or 1 or 2) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 11 (or 10) or a 16S rDNA sequence having at least 90% e.g. at least 91%, 92%, 93%, 94%, 95%, 96%, 97%, 98%, 99%; e.g. 97% or 98.7% identity thereto,
SEQ ID No. 15 (or 14) or a 16S rDNA sequence having at least 90% e.g. at least 91%, 92%, 93%, 94%, 95%, 96%, 97%, 98%, 99%; e.g. 97% or 98.7% identity thereto and
SEQ ID No. 21 or a 16S rDNA sequence having at least 90% e.g. at least 91%, 92%, 93%, 94%, 95%, 96%, 97%, 98%, 99%; e.g. 97% or 98.7% identity thereto.

9. The composition according to any preceding claim wherein said composition further comprises a pharmaceutically acceptable excipient.

10. The composition according to any preceding claim wherein said composition is formulated for oral or rectal administration.

11. The composition according to any preceding claim wherein said composition is in the form of a capsule, tablet, gel or liquid.

12. The composition according to any preceding claim wherein said composition is encapsulated in an enteric coating.

13. The composition according to any preceding claim, wherein two or more of the bacteria are in vegetative form.

14. The composition according to any preceding claim, wherein the composition comprises bacteria strains that originate from one or more human donor.

15. The composition according to any preceding claim, wherein the composition inhibits C. difficile growth and/or survival in an in vitro or in vivo assay.

16. The composition according to any preceding claim, wherein the bacteria are lyophilized.

17. The composition according to any preceding claim wherein the isolated bacterium, wherein the bacterium is a bacterium as deposited under the Budapest Treaty at DSMZ and assigned one of the following accession numbers (the date of deposit with DSMZ for each bacterium deposited is indicated in brackets after the accession number): DSM 33265 (13 Sep. 2019), DSM 33263 (13 Sep. 2019), DSM 33261 (13 Sep. 2019), DSM 33266 (13 Sep. 2019), DSM 33277 (13 Sep. 2019), DSM 33278 (13 Sep. 2019), DSM 33267 (13 Sep. 2019), DSM 33268 (13 Sep. 2019), DSM 33269 (13 Sep. 2019), DSM 33270 (13 Sep. 2019), DSM 33264 (13 Sep. 2019), DSM 33271 (13 Sep. 2019), DSM 33279 (13 Sep. 2019), DSM 33272 (13 Sep. 2019), DSM 33262 (13 Sep. 2019), DSM 33282 (13 Sep. 2019), DSM 33283 (13 Sep. 2019), DSM 33280 (13 Sep. 2019), DSM 33281 (13 Sep. 2019), DSM 33273 (13 Sep. 2019), DSM 33274 (13 Sep. 2019).

18. A method for treating or preventing a disease in a subject comprising administering a composition according to any of claims 1 to 17.

19. A composition according to any of claims 1 to 17 for use in the treatment of disease.

20. The method according to claim 18 or the composition according to claim 19 wherein the disease is a pathogenic infection.

21. The method according to claim 20 or the composition according to claim 21 wherein the pathogenic infection is selected from C. difficile, Extended spectrum beta-lactamase producing Enterobacteriaceae, Third-generation cephalosporin-resistant Enterobacteriaceae, Carbapenem-resistant Enterobacteriaceae, Fluoroquinolone-resistant Enterobacteriaceae, Salmonella spp. including S. typhi, S. paratyphi, S. enteritidis, Escherichia coli including enteroinvasive E. coli, enterohemorrhagic E. coli, Shigella toxin-producing E. coli, Klebsiella pneumoniae, Vibrio Cholerae, Campylobacter spp. including Campylobacter jejeuni, C. coli, C. lari, C. fetus, Shigella spp. including S. dysenteriae, S. flexneri, S. boydii, S. sonnei, Cryptosporidium spp., Microsporidium spp., Entamoeba histolytica, Giardia lamblia, or Blastocystis spp. including B. hominis, and combinations thereof.

22. The method according to claim 20 or the composition according to claim 19 wherein the infection is C. difficile infection.

23. The method according to any of claims 18 to 22 or the composition according to any of claims 19 to 22 wherein the C. difficile infection is a first occurrence or recurrent C. difficile infection.

24. The method according to any of claims 18 to 22 or the composition according to any of claims 19 to 29 wherein the subject has or has not been treated with antibiotics.

25. The method according to any of claims 18 to 22 or the composition according to any of claims 19 to 22 further comprising administering an antibiotic.

26. A method for increasing diversity of the gastrointestinal microbiota in a subject comprising administering a composition according to any of claims 1 to 17.

27. A method of altering the microbiota of the gastrointestinal tract in a subject comprising administering a composition according to any of claims 1 to 17.

28. A kit comprising a bacterial composition according to any of claims 1 to 17.


SEQUENCE LISTING (21 sequences)

SEQ ID NO: 1 - DNA, 1503 bp, Bacteroides cellulosilyticus
agagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatctttttgttattaaagaacttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatagtggaaacattatagccgcaaggcatttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgcaaggagcggcctagggtaaaactggtaattggggctaagtcgtaacaaggtagccgtaccggaaggtgcggctg

SEQ ID NO: 2 - DNA, 1503 bp, Bacteroides cellulosilyticus
agagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatcttcttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatagtggaaacattatagccgcaaggcatttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgcaaggagcggcctagggtaaaactggtaattggggctaagtcgtaacaaggtagccgtaccggaaggtgcggctg

SEQ ID NO: 3 - DNA, 1503 bp, Bacteroides cellulosilyticus
agagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatctttttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatctgaataatttggaaacagattagccgtaaggcagatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgtaaggagcggcctagggtaaaactggtaattggggctaagtcgtaacaaggtagccgtaccggaaggtgcggctg

SEQ ID NO: 4 - DNA, 1511 bp, Blautia sp
agagtttgatcctggctcaggatgaacgctggcggcgtgcttaacacatgcaagtcgaacgggaaatactttattgaaacttcggtggatttaatttatttctagtggcggacgggtgagtaacgcgtgggtaacctgccttatactgggggataacagccagaaatgactgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgtaatggtgggcactctgaggagactgccagggataacctggaggaaggcggggatgacgtcaaatcatcatgccccttatgatttgggctacacacgtgctacaatggcgtaaacaaagggaagcgaacccgcgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgcaagggaggagctgccgaaggcgggaccgatgactggggtgaagtcgtaacaaggtagccgtatcggaaggtgcggctg

SEQ ID NO: 5 - DNA, 1495 bp, Coprococcus catus
gtttgatcctggctcaggatgaacgctggcggcgtgcttaacacatgcaagtcgaacggacgatgaagagcttgcttttcagagttagtggcggacgggtgagtaacgcgtgggtaacctgcctcatacagggggatagcagctggaaacggctgataaaaccgcataagcgcacagcatcgcatgatgcagtgtgaaaaactccggtggtatgagatggacccgcgtctgattagctggttggtgaggtaacggcccaccaaggcgacgatcagtagccggcctgagagggtgaccggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtaggcggcggagcaagtcagaagtgaaagcccggggctcaaccccgggacggcttttgaaactgccctgcttgatttcaggagaggtaagcggaattcctagtgtagcggtgaaatgcgtagatattaggaggaacaccagtggcgaaggcggcttactggactgacaatgacgctgaggctcgaaagcgtggggagcaaacaggattagataccctggtagtccacgccgtaaacgatgaatactaggtgtcgggggccataaggctttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggccttgacatcccggtgaccgtcccgtaatggggacctctcttcggagcaccggtgacaggtggtgcatggttgtcgtcagctcgtgtcgtgagatgttgggttaagtcccgcaacgagcgcaacccctatgttcagtagccagcaggtaaggctgggcactctggacagactgccggggataacccggaggaaggcggggatgacgtcaaatcatcatgccccttacggcctgggctacacacgtgctacaatggcgtaaacaaagggaagcgagagggtgacctggagcgaatcccaaaaataacgtcccagttcggactgtagtctgcaacccgactacacgaagctggaatcgctagtaatcgcgaatcagcatgtcgcggtgaatacgttcccgggtcttgtacacaccgcccgtcacaccatgggagttggaaatgcccgaagtcagtgacctaaccgcaagggaggagctgccgaaggtggagccgatgactggggtgaagtcgtaacaaggtagccgtatcggaaggtgcggctg

SEQ ID NO: 6 - DNA, 1511 bp, Coprococcus comes
agagtttgatcctggctcaggatgaacgctggcggcgtgcttaacacatgcaagtcgaacgaagcactttaacctgattcttcggatgaaggtttttgtgactgagtggcggacgggtgagtaacgcgtgggtaacctgcctcatacagggggataacagttagaaatgactgctaataccgcataagaccacggagccgcatggctcagtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaaggagggagctgccgaaggtgggaccgataactggggtgaagtcgtaacaaggtagccgtatcggaaggtgcggctg

SEQ ID NO: 7 - DNA, 1511 bp, Dorea sp.
agagtttgatcctggctcaggatgaacgctggcggcgtgcttaacacatgcaagtcgagcgaagcacttaagtttgattcttcggatgaagacttttgtgactgagcggcggacgggtgagtaacgcgtgggtaacctgcctcatacagggggataacagttagaaatggctgctaataccgcataagaccacagtactgcatggtacagtggtaaaaactccggtggtatgagatggacccgcgtctgattaggtagttggtgaggtaacggcccaccaagccgacgatcagtagccgacctgagagggtgaccggccacattgggactgagacacggcccagactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggtagcaaagctattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccttatcttcagtagccagcatttaggatgggcactctggagagactgccagggataacctggaggaaggtggggatgacgtcaaatcatcatgccccttatgaccagggctacacacgtgctacaatggcgtaaacaaagggaagcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgaaaggagggagctgccgaaggtgggaccgataactggggtgaagtcgtaacaaggtagccgtatcggaaggtgcggctg

SEQ ID NO: 8 - DNA, 1505 bp, Unknown
  Erysipelotrichaceae UCG-003 sp.
agagtttgatcctggctcaggatgaacgctggcggcgtgcctaatacatgcaagtcgaacgcttcacttcggtgaagagtggcgaacgggtgagtaatacataagtaacctggcatctacagggggataactgatggaaacgtcagctaagaccgcataggtgtagagatcgcatgaactctatatgaaaagtgctacgggactggtagatgatggacttatggcgcattagcttgttggtagggtaacggcctaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaataggattagataccctagtagtccacgccgtaaacgatgagtactaagtgttgggggtcaaacctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatcgatctaaaggctccagagatggagagatagctatagagaagacaggtggtgcatggttgtcgtcagctcgtgtcgtgagatgttgggttaagtcccgcaacgagcgcaacccctgttgccagttgccagcattaagttggggactctggcgagactgccggtgacaagccggaggaaggcggggatgacgtcaaatcatcatgccccttatgacctgggctacacacgtgctacaatggacagagcagagggaagcgaagccgcgaggtggagcgaaacccataaaactgttctcagttcggactgcagtctgcaactcgactgcacgaagatggaatcgctagtaatcgcgaatcagcatgtcgcggtgaatacgttctcgggccttgtacacaccgcccgtcacaccatgagagtcggtaacacccgaagccggtggcctaaccgcaaggaaggagctgtctaaggtgggactgatgattggggtgaagtcgtaacaaggtatccctacgggaacgtggggatg

SEQ ID NO: 9 - DNA, 1504 bp, Odoribacter splanchnicus
agagtttgatcctggctcaggatgaacgctagcgacaggcttaacacatgcaagtcgaggggcatcatgaggtagcaataccttgatggcgaccggcgcacgggtgagtaacgcgtatgcaacctgcccgataccggggtatagcccatggaaacgtggattaacaccccatagtacttttatcctgcctgggatgggagttaaatgttcaaggtatcggatgggcatgcgtcctattagttagttggcggggtaacagcccaccaagacgatgataggtaggggttctgagaggaaggtcccccacattggaactgagacacggtccaaactcctacgggaggcagcagtgaggaatattggtcaatggacgtaagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgaaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacgggggcccgcacaagcggaggaacatgtggtttaattcgatgatacgcgaggaaccttacctgggtttaaatgggaaatgtcgtatttggaaacagatattctcttcggagcgtttttcaaggtgctgcatggttgtcgtcagctcgtgccgtgaggtgtcgggttaagtcccataacgagcgcaacccttaccgttagttgctagcatgtaatgatgagcactctaacgggactgccaccgtaaggtgagaggaaggcggggatgacgtcaaatcagcacggcccttacacccagggctacacacgtgttacaatggccggtacagagggccgctaccaggtgactggatgccaatctcaaaagccggtcgtagttcggattggagtctgtaacccgactccatgaagttggattcgctagtaatcgcgcatcagccatggcgcggtgaatacgttcccgggccttgtacacaccgcccgtcaagccatggaagccgggggtgcctgaagtccgtaaccgcgaggatcggcctagggcaaaactggtaactggggctaagtcgtaacaaggtagccgtaccggaaggtgcggctg

SEQ ID NO: 10 - DNA, 1505 bp, Parabacteroides distasonis
agagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgcgggacgtgtcctgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacagtgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtgatgctgaggactctggtgggactgccagcgtaagctgcgaggaaggcggggatgacgtcaaatcagcacggcccttacatccggggcgacacacgtgttacaatggcgtggacaaagggatgccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaaggtagccgtaccggaaggtgcggctg

SEQ ID NO: 11 - DNA, 1505 bp, Parabacteroides distasonis
agagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacagtgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggttaggctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaaggtagccgtaccggaaggtgcggctg

SEQ ID NO: 12 - DNA, 1511 bp, Ruminiclostridium 9 sp.
agagtttgatcctggctcaggatgaacgctggcggcgtgcttaacacatgcaagtcgaacggaactcctatgattgaggtttcggccaaaagaataggatgtttagtggcggacgggtgagtaacgcgtgagtaacctgccttggagtggggaataacacagtgaaaactgtgctaataccgcataatgcatttgggtcgcatgattctgaatgccaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggaaagcaagtcagatgtgaaaactgtgggctcaacccacagcctgcatttgaaactgtttttcttgagtactggagaggcagatggaattcctagtgtagcggtgaaatgcgtagatattaggaggaacaccagtggcgaaggcgatctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggcttagagataagcctttctcttcggagacactggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaagactgtgaagtggagcaaacccctaaaagccatcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctg

SEQ ID NO: 13 - DNA, 1511 bp, Ruminococcus torques
agagtttgatcctggctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctg

SEQ ID NO: 14 - DNA, 1509 bp, Bacteroides fragilis
agagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaagcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctg

SEQ ID NO: 15 - DNA, 1509 bp, Bacteroides fragilis
agagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatggcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgttagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatcttcagttactaacaggttatgctgaggactctggagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctg

SEQ ID NO: 16 - DNA, 1511 bp, Blautia sp.
agagtttgatcctggctcaggatgaacgctggcggcgtgcttaacacatgcaagtcgaacgggaaatactttattgaaacttcggtcgatttaatctatttctagtggcggacgggtgagtaacgcgtgggtaacctgccttatactgggggataacagccagaaatgactgctaataccgcataagcgcacggggccgcatggtcctgtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcgagcgttatccggatttactgggtgtaaagggagcgtagacggcgtatcaagtctgatgtgaaaggcaggggcttaacccctggactgcattggaaactggtatgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtctgggagcacagctcttaggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgacagagtatgtaatgtacttttccttcgggacaggggagacaggtggtgcatggttgtcgtcagctcgtgtcgtgagatgttgggttaagtcccgcaacgagcgcaacccctatccttagtagccagcaagtaatgttgggcactctgaggagactgccagggataacctggaggaaggcggggatgacgtcaaatcatcatgccccttatgatttgggctacacacgtgctacaatggcgtaaacaaagggaagcgaacctgtgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgtaaggaaggagctgccgaaggcgggacggatgactggggtgaagtcgtaacaaggtagccgtatcggaaggtgcggctg

SEQ ID NO: 17 - DNA, 1511 bp, Blautia sp.
agagtttgatcctggctcaggatgaacgctggcggcgtgcttaacacatgcaagtcgaacggggattatttcattgaagcttcggcagatttggtttaatcctagtggcggacgggtgagtaacgcgtgggtaacctgccttatacagggggataacagtcagaaatggctgctaataccgcataagcgcacagggccgcatggcccggtgtgaaaaactgaggtggtataagatggacccgcgttggattagccagttggcagggtaacggcctaccaaagcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcataacaagtctgatgtgaaaggctggggcttaaccccgggactgcattggaaactgttaagcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcacagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatctgcctgaccggtgagtaacgtcacctttccttcgggacaggcaagacaggtggtgcatggttgtcgtcagctcgtgtcgtgagatgttgggttaagtcccgcaacgagcgcaacccctatccccagtagccagcatgtaaaggtgggcactctgaggagactgccagggataacctggaggaaggtggggatgacgtcaaatcatcatgccccttatgatttgggctacacacgtgctacaatggcgtaaacagagggaagcgaaagggtgacctggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaaccgaaagggaggagctgccgaaggcgggacggatgactggggtgaagtcgtaacaaggtagccgtatcggaaggtgcggctg

SEQ ID NO: 18 - DNA, 1512 bp, Unknown
  Lachnospiraceae FCS020 group sp.
agagtttgatcctggctcaggatgaacgctggcggcgtgcttaacacatgcaagtcgagcgaagcacttatggagattcttcggatgatccatttgtgactgagcggcggacgggtgagtaacgcgtgagtaacctgcctcatacaggggaataacagttagaaatgactgctaatgccgcataagcgcacaggaccacatggtctggtgtgaaaaactccggtggtatgagatggactcgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggctttgcaagtctgacgtgaaaatccggggctcaaccccggaactgcgttggaaactgtgaggcttgagtgccggagaggtaagcggaattcctagtgtagcggtgaaatgcgtagatattaggaggaacaccagtggcgaaggcggcttactggacggcaactgacgttgaggctcgaaagcgtggggagcaaacaggattagataccctggtagtccacgcggtaaacgatgaatactaggtgttgggggacaaagtccttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcgattcgccgggactgtaatgagtcctttcccttcggggacgaagaagacaggtggtgcatggttgtcgtcagctcgtgtcgtgagatgttgggttaagtcccgcaacgagcgcaacccttatcttcagtagccagcaagtgaagttgggcactctggagagactgccagggacaacctggaggaaggtggggatgacgtcaaatcatcatgccccttatgacttgggctacacacgtgctacaatggcgtaaacaaagggaagcagccctgtgaaggtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggtaacgcccgaagtcagtgacccaaccgaaaggagggagctgccgaaggcgggactggtaactggggtgaagtcgtaacaaggtagccgtatcggaaggtgcggctg

SEQ ID NO: 19 - DNA, 1512 bp, Lachnoclostridium sp
agagtttgatcctggctcaggatgaacgctggcggcgtgcctaacacatgcaagtcgaacgaagcaatcagaatgaagttttcggatggatttctgattgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaactgacggtacctgactaagaagccccggctaactacgtgccagcagccgcggtaatacgtagggggcaagcgttatccggatttactgggtgtaaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgtatcggaaggtgcggctg

SEQ ID NO: 20 - DNA, 1506 bp, Bifidobacterium longum
agggttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctg

SEQ ID NO: 21 - DNA, 1509 bp, Bacteroides sp.
agagtttgatcctggctcaggatgaacgctagctacaggcttaacacatgcaagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctg